SEQUENCE LISTING (64 sequences)

SEQ ID NO: 1 - protein, 1049 aa, Listeria portnoyi
MKITKMRVDGRTIVMERTSKEGQLVYEGIDGNKTTEIIFDKKKESFYKSILNKTVRKLDEKVKDRRQRAINKAINKEIAELMLAVLHQEVPRLKLHNLKSLSTESLTKLFKPKFQNMISYPPSKDAEHVQFCLTDIAVPAIQGLDNIKPDWGVFFEKLKPYTDWVESYIHFKQTTIQRSIEQNKIQSPDSPRKVVLQKYVAAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESIVEQQFKNAVYHYVLEQGKMEAYELTNPKTKDLQDIRSGEAFSFKFINACAFASNNLKTILNPECEKDILGKGEFKKSLLNSTTRSDVVKKMIPFFSDEIQNVNFDEAIWAIRGSIQQIRNEVYHCKKHSWERILKIKDFEPNKMKYEDSDMQKLMDKDIAKIPDFIEEKLKSSGVIRFYSHDKLQSIWEMKQGFSLLTTNAPFVPSFKRVYAKGHDYQTSKNRYYDLGLTTFDILEYGEEDFRARYFLTKLVYYQQFMPWFTADNNAFRDAANFVLRLNKNRQQDAKAFINIREVEEGEMPRDYMGYVQGQIAIHEDSTEDTPNHFEKFISQVFIKGFDSHMRSADLKFIKNPRNQGLEQSEIEGMSFDIKVEPSFLKNKDDYIAFWTFCKMLDARHLSELRNEMIKYDGHLTGEQEIIGLALLGVDSRENDWKQFFSSEWEYEKIMKGYVGEELYQCEPYRQSDGKTPILFRGVEQARKYGTETVIQRLFDASPEFKVSKCNITEWERQKETIEETIKRRKELHNEWAKNPEKPQNNAFFKEYKECCDAIDAYNWHKNKTTLVYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGEKKIDNGYIETNQVSEEYCGIVKRLLEI

SEQ ID NO: 2 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 3 - skipped

SEQ ID NO: 4 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 5 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 6 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 7 - protein, 1082 aa, Prevotella sp.
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 8 - protein, 1082 aa, synthetic construct
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMK

SEQ ID NO: 9 - protein, 367 aa, Homo sapiens
SPTSPAYEPRSPGGYTPQSPSYSPTSPSYSPTSPSYSPTSPNYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPNYSPTSPNYTPTSPSYSPTSPSYSPTSPNYTPTSPNYSPTSPSYSPTSPSYSPTSPSYSPSSPRYTPQSPTYTPSSPSYSPSSPSYSPTSPKYTPTSPSYSPSSPEYTPTSPKYSPTSPKYSPTSPKYSPTSPTYSPTTPKYSPTSPTYSPTSPVYTPTSPKYSPTSPTYSPTSPKYSPTSPTYSPTSPKGSTYSPTSPGYSPTSPTYSLTSPA

SEQ ID NO: 10 - RNA, 419 nt, Encephalomyocarditis virus
  source 1..419: mol_type = genomic RNA
gtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttc

SEQ ID NO: 11 - RNA, 174 nt, Foot-and-mouth disease virus SAT 2
  source 1..174: mol_type = genomic RNA
cttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttacttt

SEQ ID NO: 12 - RNA, 432 nt, Foot-and-mouth disease virus A
  source 1..432: mol_type = genomic RNA
gtgcaatctgaaactccgcctggtctttccaggtctagaggggtgacactttgtactgtgcatgactccacgttcgatccactggcgagtgctagtagtagtactgctgtttcgtagcggaacacgacggccgtgggactccccccttggtaacaaggacccgcggggccaaaagccacgtctctgacccgtcgtgtgtgcaaacccagcacagcagctttactgcgaaaaccactttaaggtgacattgatactggtactcacgcactggtgacaggctaaggatgcccttcaggtaccccgaggcaacaagcgacactcgggatctgagaaggggactggggcttctgtaaaagcgcccagtttaaaaagcttctatgcctgaataggtgaccggaggccggcacctttccctctactaccactgactct

SEQ ID NO: 13 - RNA, 353 nt, Cricket paralysis virus
  source 1..353: mol_type = genomic RNA
ttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatca

SEQ ID NO: 14 - RNA, 174 nt, Homo sapiens
  source 1..174: mol_type = mRNA
ccgggaacccctaccccatccccttattcagcacatgaaataaacaaggggcatccaaatcttgcggcaacgcccccgggacatgcatcgtcccctggactctctcaaaccccttatccctctggacagaatgcaggtccaaccacgctggtataccctcaaacccctcagaca

SEQ ID NO: 15 - RNA, 94 nt, Homo sapiens
  source 1..94: mol_type = mRNA
cgggcccctgccgcctgcaagtcggaaattgcgctgtgctcctgtgctacggcctgtggctggactgcctgctgctgcccaactggctggcaag

SEQ ID NO: 16 - RNA, 294 nt, Homo sapiens
  source 1..294: mol_type = mRNA
gctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaacc

SEQ ID NO: 17 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 18 - protein, 1601 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTAVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 19 - RNA, 30 nt, synthetic construct
  source 1..30: mol_type = other RNA
ttctgaattcttccggatatcgcctggatc

SEQ ID NO: 20 - RNA, 30 nt, synthetic construct
  source 1..30: mol_type = other RNA
aatggccttttctgctcagtctgcccatgg

SEQ ID NO: 21 - RNA, 30 nt, synthetic construct
  source 1..30: mol_type = other RNA
tgccttaaagctgtggggcagagctacggt

SEQ ID NO: 22 - RNA, 30 nt, synthetic construct
  source 1..30: mol_type = other RNA
gatatcgcctggatcctgagccagggagga

SEQ ID NO: 23 - RNA, 30 nt, synthetic construct
  source 1..30: mol_type = other RNA
gctctccctgggcagggtggcttcgaggct

SEQ ID NO: 24 - RNA, 30 nt, synthetic construct
  source 1..30: mol_type = other RNA
gtgctcagccccaatgctgcctcctccata

SEQ ID NO: 25 - RNA, 30 nt, synthetic construct
  source 1..30: mol_type = other RNA
ggcccactaggttcccacccgaggaccagt

SEQ ID NO: 26 - RNA, 30 nt, synthetic construct
  source 1..30: mol_type = other RNA
ctgcagagagcaggggtgcaaggtcagccc

SEQ ID NO: 27 - RNA, 30 nt, synthetic construct
  source 1..30: mol_type = other RNA
tgccggggggcgggggtcaggggcgggacg

SEQ ID NO: 28 - RNA, 30 nt, synthetic construct
  source 1..30: mol_type = other RNA
acagtataaaagtaccccaaagactaaaga

SEQ ID NO: 29 - RNA, 30 nt, synthetic construct
  source 1..30: mol_type = other RNA
ccatgagggatccttgtccctgccctccct

SEQ ID NO: 30 - RNA, 30 nt, synthetic construct
  source 1..30: mol_type = other RNA
cttagttctacacctcattcattcattcag

SEQ ID NO: 31 - RNA, 30 nt, synthetic construct
  source 1..30: mol_type = other RNA
tactccatgttgcgggcagaatggggatct

SEQ ID NO: 32 - RNA, 30 nt, synthetic construct
  source 1..30: mol_type = other RNA
gggatctggacagggaagcacagggcacga

SEQ ID NO: 33 - RNA, 350 nt, synthetic construct
  source 1..350: mol_type = other RNA
ggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatca

SEQ ID NO: 34 - RNA, 414 nt, synthetic construct
  source 1..414: mol_type = other RNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgacc

SEQ ID NO: 35 - RNA, 16 nt, synthetic construct
  source 1..16: mol_type = other RNA
tcttcttttttttctc

SEQ ID NO: 36 - RNA, 15 nt, synthetic construct
  source 1..15: mol_type = other RNA
ttccctctctttccc

SEQ ID NO: 37 - RNA, 39 nt, synthetic construct
  source 1..39: mol_type = other RNA
ggagcaggcgccggatccggcgcaggagccggcgccacc

SEQ ID NO: 38 - RNA, 30 nt, synthetic construct
  source 1..30: mol_type = other RNA
gactacctctatatgaaagaggactaaaac

SEQ ID NO: 39 - RNA, 31 nt, synthetic construct
  source 1..31: mol_type = other RNA
gaccaccccaaaaatgaaggggactaaaaca

SEQ ID NO: 40 - RNA, 36 nt, synthetic construct
  source 1..36: mol_type = other RNA
caagtaaacccctaccaactggtcggggtttgaaac

SEQ ID NO: 41 - RNA, 36 nt, synthetic construct
  source 1..36: mol_type = other RNA
gttgtggaaggtccagttttgaggggctattacaac

SEQ ID NO: 42 - RNA, 16 nt, synthetic construct
  source 1..16: mol_type = other RNA
gttgatgtcacggaac

SEQ ID NO: 43 - RNA, 36 nt, synthetic construct
  source 1..36: mol_type = other RNA
caactacaaccccgtaaaaatacggggttctgaaac

SEQ ID NO: 44 - RNA, 36 nt, synthetic construct
  source 1..36: mol_type = other RNA
gaccaacacctctgcaaaactgcaggggtctaaaac

SEQ ID NO: 45 - RNA, 42 nt, synthetic construct
  source 1..42: mol_type = other RNA
ttgacgcggttctatctagttacgcgttaaaccaactagaaa

SEQ ID NO: 46 - RNA, 51 nt, synthetic construct
  source 1..51: mol_type = other RNA
gggtcaggagccccccccctgaacccaggataaccctcaaagtcggggggc

SEQ ID NO: 47 - RNA, 60 nt, synthetic construct
  source 1..60: mol_type = other RNA
gtcagggtcaggagccccccccctgaacccaggataaccctcaaagtcggggggcaaccc

SEQ ID NO: 48 - RNA, 56 nt, synthetic construct
  source 1..56: mol_type = other RNA
gggtcaggagccccccccctgaacccaggaaaaccctcaaagtcggggggcaaccc

SEQ ID NO: 49 - RNA, 56 nt, synthetic construct
  source 1..56: mol_type = other RNA
gggtcaggagccccccccctgcacccaggaaaaccctcaaagtcggggggcaaccc

SEQ ID NO: 50 - RNA, 21 nt, synthetic construct
  source 1..21: mol_type = other RNA
taatttctactaagtgtagat

SEQ ID NO: 51 - RNA, 42 nt, synthetic construct; identical to SEQ ID NO: 45
  source 1..42: mol_type = other RNA

SEQ ID NO: 52 - RNA, 21 nt, synthetic construct
  source 1..21: mol_type = other RNA
taagtctccatagaatggagg

SEQ ID NO: 53 - RNA, 20 nt, synthetic construct
  source 1..20: mol_type = other RNA
taatttctactcttgtagat

SEQ ID NO: 54 - RNA, 19 nt, synthetic construct
  source 1..19: mol_type = other RNA
gggccctgaagaagggccc

SEQ ID NO: 55 - RNA, 63 nt, synthetic construct
  source 1..63: mol_type = other RNA
ggaggtcagggtcaggagcccccccctgaacccaggataaccctcaaagtcggggggcaaccc

SEQ ID NO: 56 - RNA, 55 nt, synthetic construct
  source 1..55: mol_type = other RNA
gggtcaggagcccccccctgaacccaggataaccctcaaagtcggggggcaaccc

SEQ ID NO: 57 - RNA, 34 nt, synthetic construct
  source 1..34: mol_type = other RNA
ggccaacatgaggatcacccatgtctgcagggcc

SEQ ID NO: 58 - RNA, 29 nt, synthetic construct
  source 1..29: mol_type = other RNA
ccggagcagacgatatggcgtcgctccgg

SEQ ID NO: 59 - RNA, 26 nt, synthetic construct
  source 1..26: mol_type = other RNA
ccaaaggctcttctcagagccaccca

SEQ ID NO: 60 - RNA, 31 nt, synthetic construct
  source 1..31: mol_type = other RNA
ggccagatctgagcctgggagctctctggcc

SEQ ID NO: 61 - RNA, 36 nt, synthetic construct
  source 1..36: mol_type = other RNA
atataaccttcaccattaggttcaaataatggtaat

SEQ ID NO: 62 - RNA, 42 nt, synthetic construct
  source 1..42: mol_type = other RNA
cgcctcccgggttcaagcgattctcctgcctcagcctcccga

SEQ ID NO: 63 - protein, 7 aa, synthetic construct
YSPTSPS

SEQ ID NO: 64 - DNA, 360 bp, synthetic construct
  source 1..360: mol_type = other DNA
gttaatcgaattgaactgaaaggtattgattttaaggaggacggtaacatactggggcacaagttggagtacaactttaacagccataatgtgtatattaccgctgataagcagaaaaatgggataaaggccaactttaagatccgacataatgtcgaagatggtagtgttcaactggctgatcattaccaacaaaatacgcccatcggagatggacctgtactcttgcctgacaatcattatctctccacgcaatcaaagctttccaaggacccaaacgaaaagagagatcacatggtccttctggaatttgtgactgccgcaggcatcactctcggtatggatgagctgtacaagtga

(19) United States
(12) Patent Application Publication
(10) Pub. No.: US 2025/0283112 A1
(43) Pub. Date: Sep. 11, 2025

(54) Compositions and Methods for RNA Editing

(71) Applicant: The Regents of the University of California, Oakland, CA (US)

(72) Inventors: Patrick Hsu, Berkeley, CA (US); Sita Chandrasekaran, Berkeley, CA (US); Cyrus Tau, Berkeley, CA (US)

(21) Appl. No.: 18/863,599

(22) PCT Filed: May 11, 2023
(86) PCT No.: PCT/US2023/066872

§ 371 Date: Nov. 6, 2024

Related U.S. Application Data
(60) Provisional application No. 63/341,823, filed on May 13, 2022.

(51) Int. Cl. C12N 15/87; C12N 9/22; C12N 15/113

(52) U.S. Cl. CPC C12N 15/87; C12N 9/226; C12N 15/113; C12N 2310/14; C12N 2310/20; C12N 2310/3519

(57) ABSTRACT

The present disclosure provides a hybrid RNA molecule comprising a targeting region and a donor RNA, and compositions comprising the hybrid RNA molecule. A hybrid RNA molecule of the present disclosure is useful in methods of modifying a target RNA, which methods are also provided.

### CROSS-REFERENCE

This application claims the benefit of U.S. Provisional Patent Application No. 63/341,823, filed May 13, 2022, which application is incorporated herein by reference in its entirety.

### STATEMENT REGARDING FEDERALLY SPONSORED RESEARCH

This invention was made with government support under Grant Nos. GM131073, GM132465, and OD021369, awarded by the National Institutes of Health. The government has certain rights in the invention.

### INCORPORATION-BY-REFERENCE OF MATERIAL ELECTRONICALLY SUBMITTED

A Sequence Listing is provided herewith as a Sequence Listing XML, “BERK-465WO_SEQ_LIST” created on May 10, 2023 and having a size of 70,860 bytes. The contents of the Sequence Listing XML are incorporated by reference herein in their entirety.

### INTRODUCTION

RNA splicing is a fundamental biological process, in which a pre-mRNA transcript is modified by the endogenous spliceosome into a mature mRNA transcript. This standard process involves a single pre-mRNA molecule “in cis.” Some organisms, such as trypanosomes, flatworms, and nematodes, can employ a splicing reaction that joins two distinct RNA molecules “in trans.”

Methods for editing DNA using editing enzymes have been described and are currently in use for various gene editing applications. There is a need in the art for methods of editing RNA.

### SUMMARY

The present disclosure provides a hybrid RNA molecule comprising a targeting region and a donor RNA, and compositions comprising the hybrid RNA molecule. A hybrid RNA molecule of the present disclosure is useful in methods of modifying a target RNA, which methods are also provided.

### BRIEF DESCRIPTION OF THE DRAWINGS

FIG. 1A-1B are a schematic representation of a pathway of cis-splicing (FIG. 1A) and a pathway of trans-splicing (FIG. 1B).

FIG. 2A-2C schematically depict targeting of a trans-splicing molecule to a transcript of interest (FIG. 2A), interfering with cis-splicing using an RNA targeting protein (FIG. 2B), and guiding a trans-splicing molecule with an RNA targeting protein (FIG. 2C).

FIG. 3 schematically depicts enhanced localization of a gTSM (a trans splicing molecule (TSM) fused to a guide RNA) to actively transcribed and spliced transcripts using a CTD interacting domain (CID) fused to an RNA targeting protein.

FIG. 4 schematically depicts enhanced localization of a gTSM to its target through an RS domain fusion to the RNA targeting protein.

FIG. 5A-5C schematically depict addition of an RNA nuclear localization sequence to the gTSM (FIG. 5A), inclusion of a guided interfering molecule to the gTSM to interrupt cis-splicing (FIG. 5B), and inclusion of a guided interfering molecule targeting DNA to interrupt RNA transcription downstream of the targeted site, thereby reducing the number of transcripts that are able to splice in cis (FIG. 5C).

FIG. 6A-6C schematically depict 3′ trans-splicing (FIG. 6A), 5′ trans-splicing (FIG. 6B), and internal trans-splicing (FIG. 6C).

FIG. 7A-7B schematically depict a mechanism of Huntington's disease (FIG. 7A) and use of trans-splicing to correct Huntington's disease (FIG. 7B).

FIG. 8A-8B schematically depict a mechanism of spinal muscular atrophy (FIG. 8A) and use of trans-splicing to correct spinal muscular atrophy (FIG. 8B).

FIG. 9A-9B schematically depict plasmids transfected into HEK293 cells (FIG. 9A) and various gTSM locations (FIG. 9B).

FIG. 10 depicts the results of transfection of HEK293 cells with the plasmids depicted in FIG. 9A-9B.

FIG. 11A-11H provide amino acid sequences of various CRISPR-Cas effector polypeptides (SEQ ID NOs:1-8; respectively).

FIG. 12 provides the amino acid sequence of a C-terminal domain polypeptide of RNA Polymerase II (SEQ ID NO:9).

FIG. 13A-13D present schematic depictions of exemplary embodiments of hybrid RNA molecules of the present disclosure.

FIG. 14 presents schematic depictions of exemplary embodiments of hybrid RNA molecules of the present disclosure.

FIG. 15A-15G provide examples of suitable internal ribosome entry sites (IRES) (SEQ ID NOs:10-16; respectively).

FIG. 16 provides the amino acid sequence of a Cas7-11 polypeptide (SEQ ID NOs:17).

FIG. 17 provides the amino acid sequence of a dCas7-11 polypeptide (SEQ ID NOs:18).

FIG. 18 provides non-limiting examples of targeting region sequences that can be included in a hybrid RNA molecule of the present disclosure (SEQ ID NOs:19-32 and 19; respectively).

FIG. 19 provides non-limiting examples of diseases that can be treated using a method of the present disclosure.

FIG. 20 depicts the effect of catalytically active Cas7-11 on trans-splicing.

FIG. 21A-21E depict trans-splicing with dCasRx as the effector.

FIG. 22A-22B depict targeting TDP43 (FIG. 22A) or SMARCA4 (FIG. 22B) with hit gTSM and testing gIMs.

FIG. 23 schematically depicts use of gTSM and gIM to target the intron between exon 7 and 8 of a target transcript.

FIG. 24 depicts the percent trans-splicing efficiency for ITGB1, as measured by ddPCR.

FIG. 25 depicts the percent trans-splicing efficiency for ITGB1, as measured by ddPCR using four different RNA-targeting CRISPR effectors for the gTSM (dCasRx, dCasRx-RBD fusion, HifiCasRx, HifiCasRx-RBD).

### DEFINITIONS

“Heterologous,” as used herein in the context of an RNA, refers to a nucleotide sequence that is not found in the native nucleic acid. For example, a donor RNA is generally heterologous to the targeting region present in a hybrid RNA molecule of the present disclosure. “Heterologous,” as used herein in the context of a polypeptide, refers to an amino acid sequence that is not found in the native polypeptide. For example, a fusion CRISPR-Cas effector polypeptide comprises: a) a CRISPR-Cas effector polypeptide; and b) one or more heterologous polypeptides, where the heterologous polypeptide comprises an amino acid sequence from a protein other than a CRISPR-Cas effector polypeptide.

“Recombinant,” or “hybrid,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. For example, a hybrid RNA molecule of the present disclosure can be generated by either chemical synthesis means, or by the artificial manipulation of isolated segments of nucleic acids, e.g., by genetic engineering techniques.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Cap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive expression of the various nucleic acids of the present disclosure.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

The term “induced pluripotent stem cell” or “iPSC” refers to a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

The term “somatic cell” refers to any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

The term “mitotic cell” refers to a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

The term “post-mitotic cell” refers to a cell that has exited from mitosis, i.e., it is “quiescent”, i.e. it is no longer undergoing divisions. This quiescent state may be temporary, i.e. reversible, or it may be permanent.

The term “meiotic cell” refers to a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a hybrid RNA molecule” includes a plurality of such molecules and reference to “the secondary structure” includes reference to one or more secondary structures and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a hybrid RNA molecule comprising a targeting region and a donor RNA, and compositions comprising the hybrid RNA molecule. A hybrid RNA molecule of the present disclosure is useful in methods of modifying a target RNA, which methods are also provided.

### Hybrid RNA Molecules

The present disclosure provides a hybrid RNA molecule comprising: a) a targeting region that comprises a nucleotide sequence that is complementary to a target sequence of a target RNA (“targeting region”); and b) a donor RNA (“donor region” or “donor sequence”).

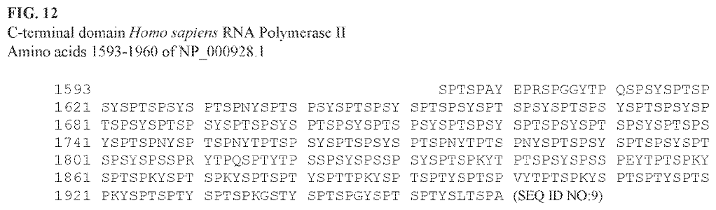

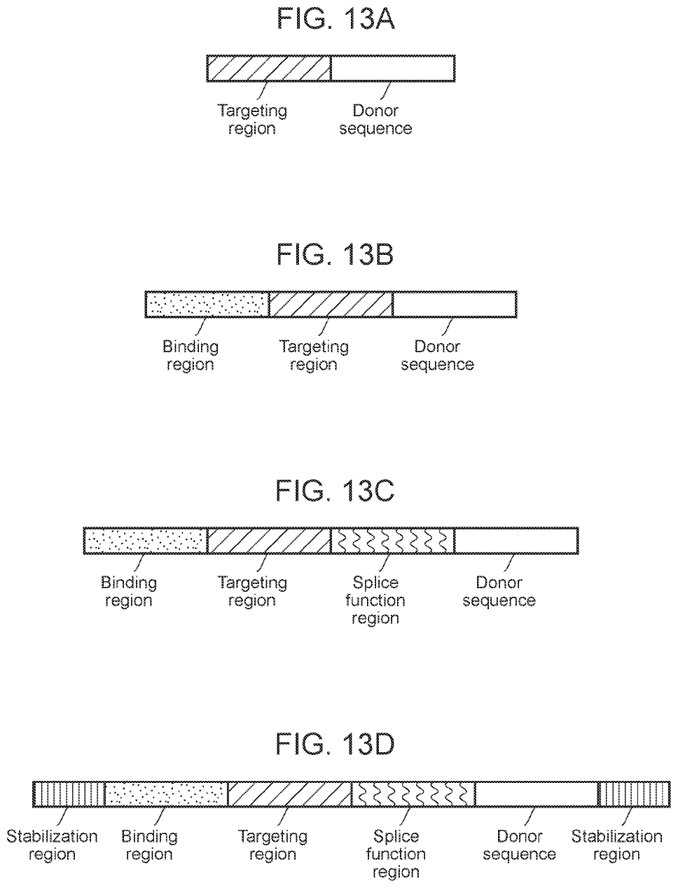

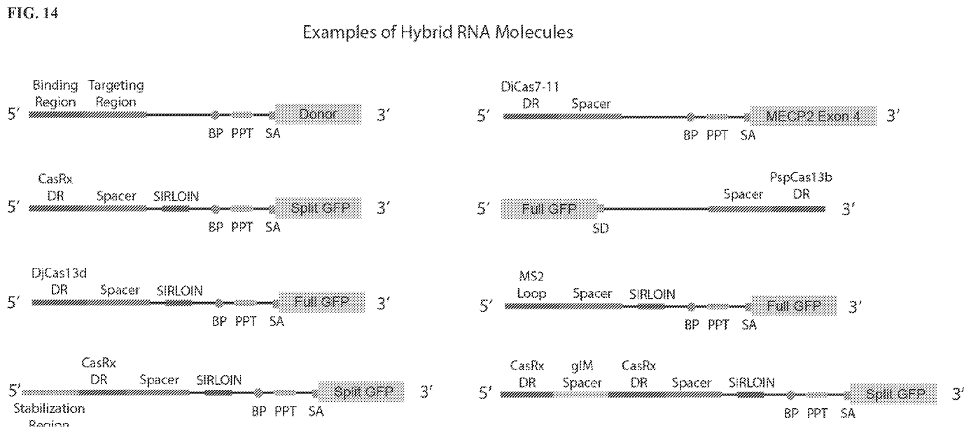

A hybrid RNA molecule will in some cases comprise, in addition to a targeting region and a donor RNA, one or more of: (i) a binding region that binds to an RNA binding or modifying polypeptide (“binding region”); (ii) a protein-recruiting region; (iii) a splice function region; and (iv) a stabilization region. FIG. 13A-13D provide schematic depictions of various exemplary, non-limiting embodiments of a hybrid RNA molecule of the present disclosure. For example, in some cases, a hybrid RNA molecule comprises: (a) a targeting region; and (b) a donor sequence, as depicted schematically in FIG. 13A. As another example, in some cases, a hybrid RNA molecule comprises: (a) a targeting region; (b) a donor sequence; and (c) a binding region, as depicted schematically in FIG. 13B. As another example, in some cases, a hybrid RNA molecule comprises: (a) a targeting region; (b) a donor sequence; (c) a binding region; and (d) a splice function region, as depicted schematically in FIG. 13C. As another example, in some cases, a hybrid RNA molecule comprises: (a) a targeting region; (b) a donor sequence; (c) a binding region; (d) a splice function region; and (e) one or more stabilization regions, as depicted schematically in FIG. 13D. Any of these embodiments may further include a protein recruiting region.

In some cases, the targeting region and the donor region are in the same RNA molecule; e.g., some cases, the targeting region and the donor region are linked via a phosphodiester bond, or another type of covalent bonds, such as a phosphorothioate linkage, in the same RNA molecule. Alternatively, the targeting region and the donor region can be in two separate RNA molecules that are non-covalently associated with one another, where the non-covalent association can be via: i) hybridization of a stretch of contiguous nucleotides in a first RNA molecule comprising the targeting region with a stretch of contiguous nucleotides in a second RNA molecule comprising the donor region; ii) binding of an avidin moiety present in a first RNA molecule comprising the targeting region with a biotin moiety present in a second RNA molecule comprising the donor region. As another alternative, a first RNA molecule comprising the targeting region and a second RNA molecule comprising the donor region can be covalently linked to one another via a linkage other than a phosphodiester bond, e.g., where the linkage is via a click chemistry reaction (se, e.g., Fantoni et al. (2021) Chem. Rev. 12:7122).

### Targeting Region

As noted above, a hybrid RNA molecule of the present disclosure comprises a targeting region that comprises a nucleotide sequence that is complementary to a target sequence of a target RNA. The nucleotide sequence that is complementary to a target sequence of a target RNA can have a length of from about 10 nucleotides (nt) to about 200 nt (e.g., from about 10 nt to about 15 nt, from about 15 nt to about 20 nt, from about 20 nt to about 25 nt, from about 25 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, from about 90 nt to about 100 nt, from about 100 nt to about 125 nt, from about 125 nt to about 150 nt, from about 150 nt to about 175 nt, or from about 175 nt to about 200). In some cases, the nucleotide sequence that is complementary to a target sequence of a target RNA has a length of from about 10 nt to about 20 nt. In some cases, the nucleotide sequence that is complementary to a target sequence of a target RNA has a length of from about 10 nt to about 15 nt. In some cases, the nucleotide sequence that is complementary to a target sequence of a target RNA has a length of from about 15 nt to about 20 nt. In some cases, the nucleotide sequence that is complementary to a target sequence of a target RNA has a length of from about 10 nt to about 50 nt. In some cases, the nucleotide sequence that is complementary to a target sequence of a target RNA has a length of from about 25 nt to about 50 nt. In some cases, the nucleotide sequence that is complementary to a target sequence of a target RNA has a length of from about 50 nt to about 100 nt. In some cases, the nucleotide sequence that is complementary to a target sequence of a target RNA has a length of from about 100 nt to about 200 nt.

The nucleotide sequence that is complementary to a target sequence of a target RNA will in some cases have 100% complementarity to the target sequence, e.g., 100% complementarity over the same number of contiguous nucleotides. For example, if the nucleotide sequence that is complementary to a target sequence of a target RNA has a length of 20 nt, in some cases, the target sequence in the target RNA will have a length of 20 nt and in some cases, the nucleotide sequence that is complementary to the target sequence will have 100% complementarity to the target sequence.

In other cases, the nucleotide sequence that is complementary to the target sequence in a target RNA will have less than 100% complementarity to a target sequence of a target RNA, e.g., the nucleotide sequence that is complementary to the target sequence will have 1, 2, 3, 4, or 5 mismatches compared to the target sequence. In some cases, the nucleotide sequence that is complementary to the target sequence in a target RNA will have a single mismatch to the target sequence. In some cases, the nucleotide sequence that is complementary to the target sequence in a target RNA will have 2 mismatches to the target sequence. In some cases, the nucleotide sequence that is complementary to the target sequence in a target RNA will have 3 mismatches to the target sequence. In some cases, the nucleotide sequence that is complementary to the target sequence in a target RNA will have 4 mismatches to the target sequence. In some cases, the nucleotide sequence that is complementary to the target sequence in a target RNA will have 5 mismatches to the target sequence.

The target sequence in the target RNA can be any sequence of interest. For example, the target sequence in the target RNA can be: (i) a nucleotide sequence in an exon (e.g., a nucleotide sequence encoding all or a portion of a polypeptide); (ii) a nucleotide sequence in a 5′ splice sequence; (iii) a nucleotide sequence in a 3′ splice sequence; (iv) a nucleotide sequence in an intron; (v) a nucleotide sequence in a regulatory region of a target RNA; (vi) a nucleotide sequence forming a secondary structure in a target RNA; (vii) a nucleotide sequence in a protein-binding site of an RNA; and the like.

### Donor RNA

As noted above, a hybrid RNA molecule of the present disclosure comprises a donor RNA. The donor RNA comprises a nucleotide sequence that will be inserted into a target RNA or that will replace a portion of a target RNA.

In some cases, a donor RNA has a length of from 1 nucleotide (nt) to 9000 nt; e.g., the donor RNA can have a length of from 1 nt to 10 nt, from 10 nt to 15 nt, from 15 nt to 20 nt, from 20 nt to 25 nt, from 25 nt to 30 nt, from 30 nt to 40 nt, from 40 nt to 50 nt, from 50 nt to 60 nt, from 60 nt to 70 nt, from 70 nt to 80 nt, from 80 nt to 90 nt, from 90 nt to 100 nt, from 100 nt to 250 nt, from 250 nt to 500 nt, from 500 nt to 1000 nt, from 1000 nt to 2000 nt, from 2000 nt to 3000 nt, from 3000 nt to 4000 nt, from 4000 nt to 5000 nt, from 5000 nt to 6000 nt, from 6000 nt to 7000 nt, from 7000 nt to 8000 nt, or from 8000 nt to 9000 nt. In some cases, a donor RNA has a length greater than 8000 nt. In some cases, a donor RNA has a length of 1 nt, 2 nt, 3 nt, 4 nt 5 nt, 6 nt, 7 nt, 8 nt, 9 nt, or 10 nt. In some cases, a donor RNA has a length of from 10 nt to 20 nt (e.g., 10, 11, 12, 13, 14, 15, 16, 17, 18, 19, or 20 nt). In some cases, a donor RNA has a length of from 20 nt to 30 nt (e.g., 20, 21, 22, 23, 24, 25, 26, 27, 28, 29, or 30 nt). In some cases, a donor RNA has a length of from 20 nt to 100 nt. In some cases, a donor RNA has a length of from 100 nt to 200 nt. In some cases, a donor RNA has a length of from 100 nt to 250 nt. In some cases, a donor RNA has a length of from 100 nt to 500 nt. In some cases, a donor RNA has a length of from 100 nt to 1000 nt. In some cases, a donor RNA has a length of from 1000 nt to 1500 nt. In some cases, a donor RNA has a length of from 1500 nt to 2000 nt. In some cases, a donor RNA has a length of from 1500 nt to 2500 nt. In some cases, a donor RNA has a length of from 2000 nt to 8000 nt. In some cases, a donor RNA has a length of from 2000 nt to 7000 nt. In some cases, a donor RNA has a length of from 2000 nt to 6000 nt. In some cases, a donor RNA has a length of from 2000 nt to 5000 nt.

The donor RNA can include one or more of: a) a coding region comprising a nucleotide sequence encoding all or a portion of a polypeptide; b) an untranslated region (UTR); c) a bar code (i.e., a nucleotide sequence that is unique to the donor RNA that distinguishes it from other nucleic acids); d) an internal ribosomal entry site (IRES); e) a poly(pyrimidine) tract (e.g., a poly(A) tract or a poly(U) tract); f) an RNA response element; g) an intron; h) an aptamer (e.g., a fluorescent aptamer).

### Coding Region

In some cases, the donor RNA comprises a nucleotide sequence encoding all or a portion of a polypeptide. For example, in some cases, the donor RNA comprises a nucleotide sequence encoding from 1 to 3000 contiguous amino acids (e.g., from 1 to 5, from 5 to 10, from 10 to 15, from 15 to 20, from 20 to 25, from 25 to 30, from 30 to 40, from 40 to 50, from 50 to 60, from 60 to 70, from 70 to 80, from 80 to 90, from 90 to 100, from 100 to 150, from 150 to 200, from 200 to 250, from 250 to 300, from 300 to 400, from 400 to 500, from 500 to 1000, from 1000 to 1250, from 1250 to 1500, from 1500 to 2000, from 2000 to 2500, or from 2500 to 3000, contiguous amino acids). In some cases, the polypeptide, or portion thereof, encoded by a donor RNA is a non-diseased form of a diseased protein (or portion thereof). In some cases, the polypeptide, or portion thereof, encoded by a donor RNA, is a functional form of a non-functional protein (or portion thereof). In some cases, the polypeptide, or portion thereof, encoded by a donor RNA is a fluorescent protein, thus adding a fluorescent tag to the protein encoded by the target RNA. Suitable fluorescent proteins include, but are not limited to, a green fluorescent protein, a yellow fluorescent protein, a blue fluorescent protein, and a red fluorescent protein. In some cases, the polypeptide, or portion thereof, encoded by a donor RNA, is an epitope tag, thus enabling affinity purification of the protein encoded by the target RNA. Suitable epitope tags include, but are not limited to, a FLAG tag, a hemagglutinin (HA), a SNAP tag, a c-Myc tag, and a HALO tag. In some cases, the polypeptide, or portion thereof, encoded by a donor RNA, is an effector polypeptide, thus fusing an effector polypeptide to the protein encoded in the target RNA. Suitable effector polypeptides include, but are not limited to, polypeptides that can modify a nucleotide or ribonucleotide (e.g., a cytidine deaminase, a pentatricopeptide repeat (PPR) protein, an adenosine deaminase, an adenosine deaminase acting on RNA (ADAR) family protein, or an APOBEC family protein); polypeptides that can enable proximity-based protein labeling and identification (e.g., a biotin ligase (such as BirA) or a peroxidase (such as APEX2) in order to biotinylate proteins that interact with the protein encoded by the target RNA); RNA editing enzymes (e.g., RNA deaminases, e.g., ADAR polypeptides, including A to I and/or C to U editing enzymes); helicases; RNA-binding proteins; and the like.

### IRES

As noted above, in some cases, the donor RNA present in a hybrid RNA molecule of the present disclosure includes one or more (e.g., 1, 2, 3, 4, or 5, or more) internal ribosome entry sites (IRES). IRES elements are able to bypass the ribosome scanning model of 5′ methylated cap-dependent translation and begin translation at internal sites. Thus, an IRES provides a structure to which a ribosome can bind that does not need to be at the 5′ end of the mRNA. An IRES can therefore allow a ribosome to initiate translation at a second (or additional) initiation codon within a mRNA, allowing more than one polypeptide to be produced from a single mRNA.

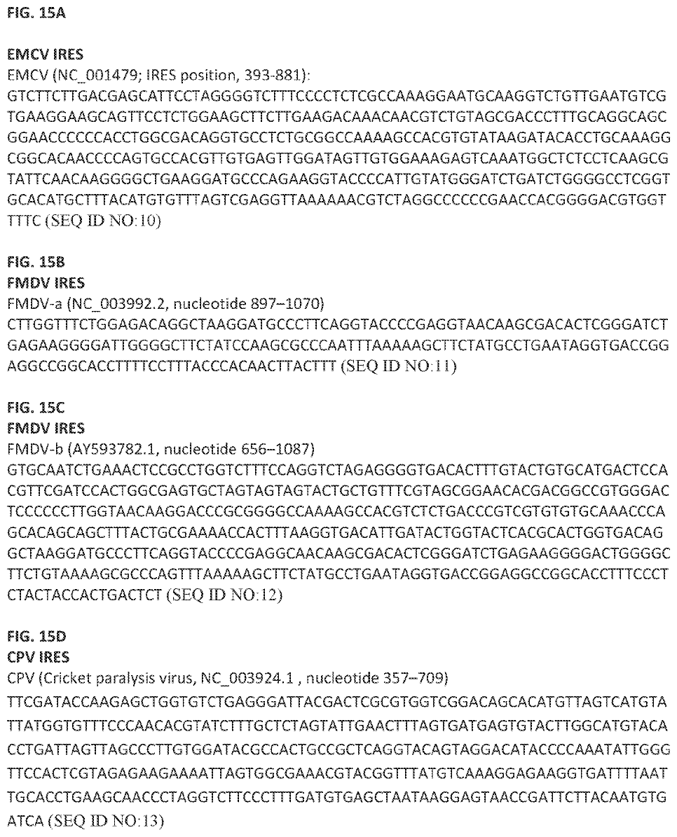

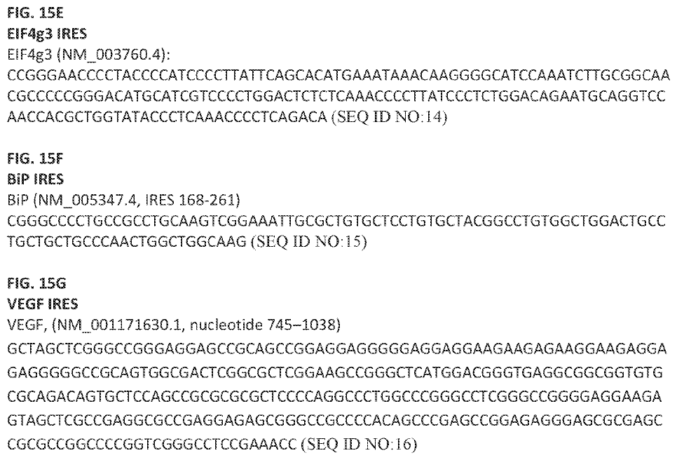

Suitable IRESs include, but are not limited to, a picornavirus IRES, an Aphthovirus IRES, a Kaposi's sarcoma-associated herpesvirus IRES, a hepatitis A virus IRES, a hepatitis C virus IRES, a pestivirus IRES, a cripavirus IRES, a Friend murine leukemia virus IRES, a Moloney murine leukemia virus IRES, a Rouse sarcoma virus IRES, a human immunodeficiency virus IRES, a mammalian IRES, and the like. In some cases, the IRES is an IRES from a picornavirus (e.g. a pest viruses (CFFV), a polio virus (PV), an encephalomyocarditis virus (ECMV), a foot-and-mouth disease virus (FMDV), a hepatitis C virus (HCV), a classical swine fever virus (CSFV), a murine leukemia virus (MLV), a simian immune deficiency virus (SIV), or a cricket paralysis viruses (CPV). An IRES can be derived from a eukaryotic non-translated region, e.g., from BiP, fibroblast growth factor-1 (FGF-1), FGF-2, and the like. An IRES can include a naturally-occurring sequence. An IRES can be a synthetic IRES (e.g., comprising a nucleotide sequence not found in nature). Non-limiting examples of IRES sequences are provided in FIG. 15A-15G.

In some cases, the donor sequence comprises an eToehold. See, e.g., Zhao et al. (2021) Nature Biotechnol. 40:539.

In some cases, the donor RNA comprises a nucleotide sequence that provides a functional 3′ splice site. In some cases, the donor RNA comprises a nucleotide sequence that provides a functional 5′ splice site. In some cases, the donor RNA comprises a nucleotide sequence that is all or a portion of an intron. Any of these components can comprises a naturally-occurring nucleotide sequence, or can be a synthetic (non-naturally-occurring) sequence.

### Splice Function Region

In some cases, a hybrid RNA molecule of the present disclosure comprises a splice function region. A splice function region can include one or more of: a) a splice modifying sequence; b) a splice exclusion sequence; c) an intron; d) a branch point; e) an intronic splice enhancer; f) an intronic splice silencer; g) a poly(pyrimidine tract); and h) a SIRLOIN. Any of these components can comprises a naturally-occurring nucleotide sequence, or can be a synthetic (non-naturally-occurring) sequence.

Branch points map within short motifs upstream of acceptor splice sites and are essential for splicing of pre-mature mRNA. Branch points are known in the art; see, e.g., Leman et al. (2020) BMC Genomics 21:86. As one non-limiting example, a branch point can comprise the sequence: TGCTAAC. As another non-limiting example, a branch point can comprise the sequence: CTGAT.

Suitable poly(pyrimidine) tracts include, e.g., TCTTCTTTTTTTTCTC (SEQ ID NO:35); and TTCCCTCTCTTTCCC (SEQ ID NO:36).

In some cases, a hybrid RNA molecule comprises a “stuffer” sequence that separates the targeting region from the splice function region and/or that separates the splice function region and the donor sequence. A suitable “stuffer” sequence is as follows:

(SEQ ID NO: 37)
GGAGCAGGCGCCGGATCCGGCGCAGGAGCCGGCGCCACC.

### Binding Region

As noted above, in some cases, a hybrid RNA molecule includes a binding region, e.g., a stretch of nucleotides that binds to an RNA targeting polypeptide such as an RNA-targeting Clustered Regularly Interspaced Short Palindromic Repeat (CRISPR)-associated (CRISPR-Cas) polypeptide (also referred to herein as a “CRISPR-Cas effector polypeptide”). Suitable RNA-targeting CRISPR-Cas effector polypeptides include Type II CRISPR-Cas effector polypeptides, Type III CRISPR Cas effector polypeptides, and Type VI CRISPR-Cas effector polypeptides. Examples of Type TT CRISPR-Cas polypeptides include Cas9 polypeptides, e.g., Staphylococcus aureus Cas9, Streptococcus pyogenes Cas9, etc. Examples of Type VI CRISPR-Cas effector polypeptides include a Cas13a polypeptide, a Cas13b polypeptide, a Cas13c polypeptide, a Cas13d polypeptide, a Cas13e polypeptide, a Cas13f polypeptide, a Cas13X polypeptide, and a Cas13Y polypeptide. A suitable Type III CRISPR-Cas effector polypeptide is a Cas7-11 polypeptide (see, e.g., Ozcan et al. (2021) Nature 597:710). A suitable CRISPR-Cas effector polypeptide is an RNA-binding CRISPR-Cas effector polypeptide.

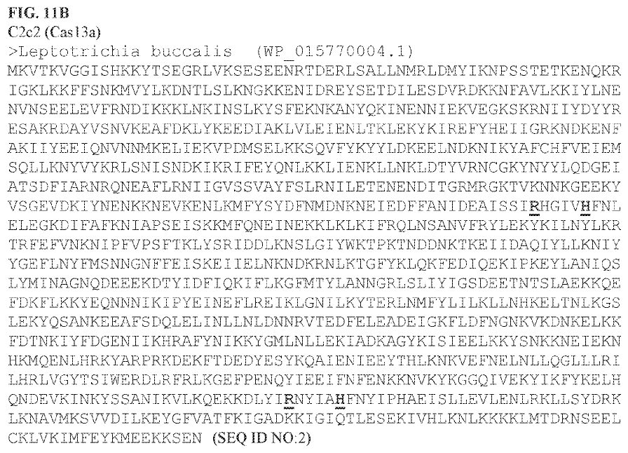

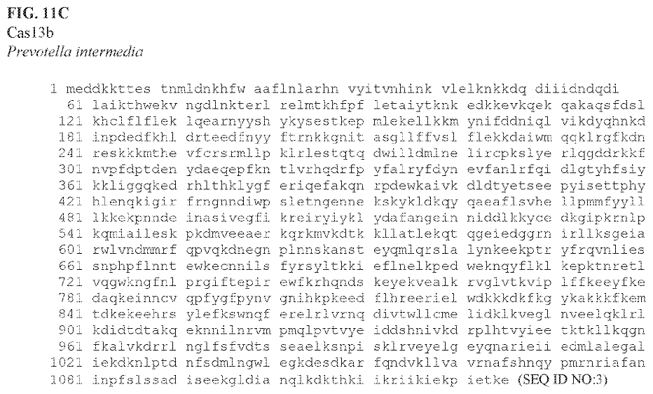

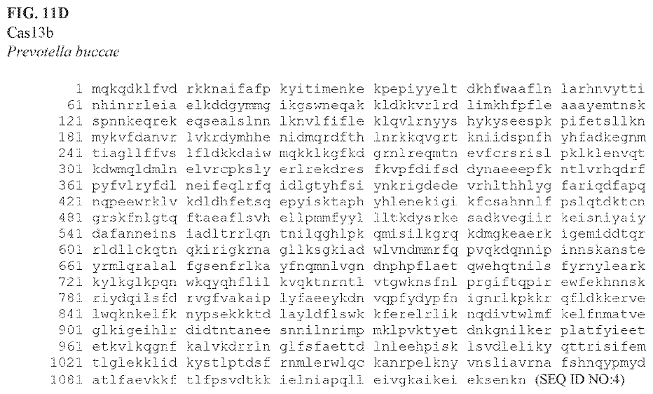

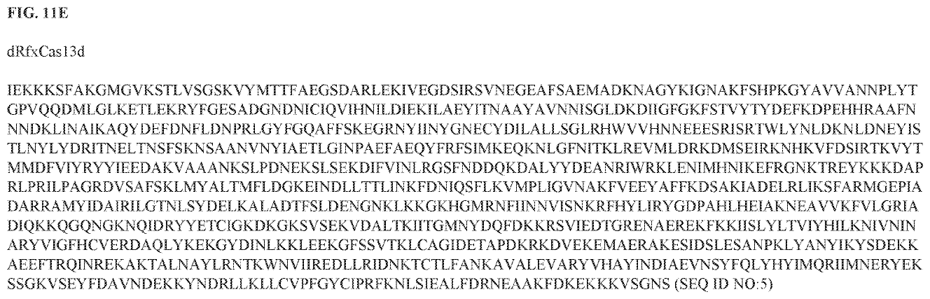

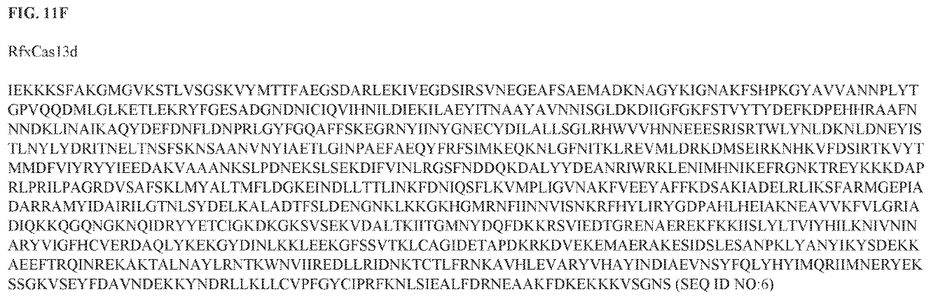

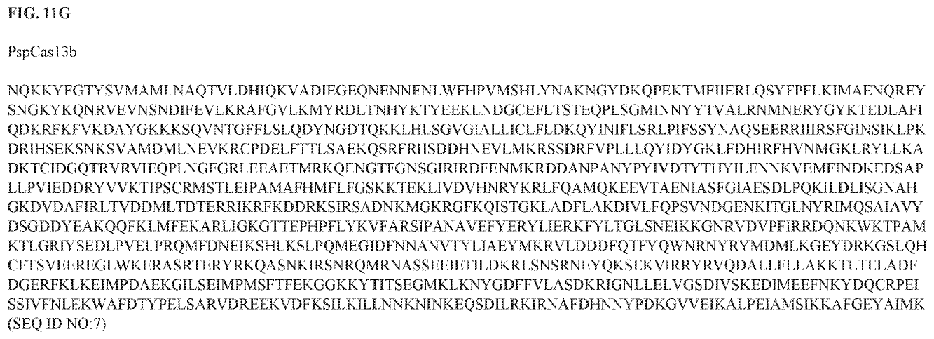

For example, in some cases, the CRISPR-Cas effector polypeptide is an RFx Cas13d polypeptide (see FIG. 11F). As another example, in some cases, the CRISPR-Cas effector polypeptide is a dRfxCas13d polypeptide (see FIG. 11E). As another example, in some cases, the CRISPR-Cas effector polypeptide is an enhanced RfxCas13d that cleaves target RNA with minimal collateral degradation of off-target RNAs (see, eg. Tong et al. (2022) Nature Biotechnology 41.1 (2023): 108-119). As another example, in some cases, the CRISPR-Cas effector polypeptide is an RfxCas13d fused to a dsRNA binding domain to increase specificity (see, e.g. Han et al. (2020) PNAS 117.36 (2020): 22068-22079).

As another example, in some cases, the CRISPR-Cas effector polypeptide is a DjCas13d polypeptide. As another example, in some cases, the CRISPR-Cas effector polypeptide is a PspCas13b polypeptide, e.g., Prevotella sp. P5-125 Cas13b (see FIG. 11G). As another example, in some cases, the CRISPR-Cas effector polypeptide is a dPspCas13b polypeptide (see FIG. 11H). In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to any of the amino acid sequences depicted in FIG. 11A-11H. In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 11F. In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 11E. In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 11G. In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 11H.

In some cases, a Cas13 polypeptide (e.g., a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide) comprises two Higher Eukaryotes and Prokaryotes Nucleotide-binding (HEPN) domains, each comprising a HEPN motif, where each HEPN motif is RXXXXH, RXXXXXH, or RXXXXXXH, where X is any amino acid.

In some cases, the CRISPR-Cas effector polypeptide is a variant that exhibits reduced catalytic activity compared to a wild-type CRISPR-Cas effector polypeptide. For example, where the CRISPR-Cas effector polypeptide (e.g., a Cas13 polypeptide) comprises a HEPN domain, the CRISPR-Cas effector polypeptide can comprise a substitution of the Arg and/or the His in the HEPN motif, where such a mutation reduces the catalytic activity of the CRISPR-Cas effector polypeptide. In some cases, the RNA-binding CRISPR-Cas effector polypeptide binds, but does not cleave, a target RNA.

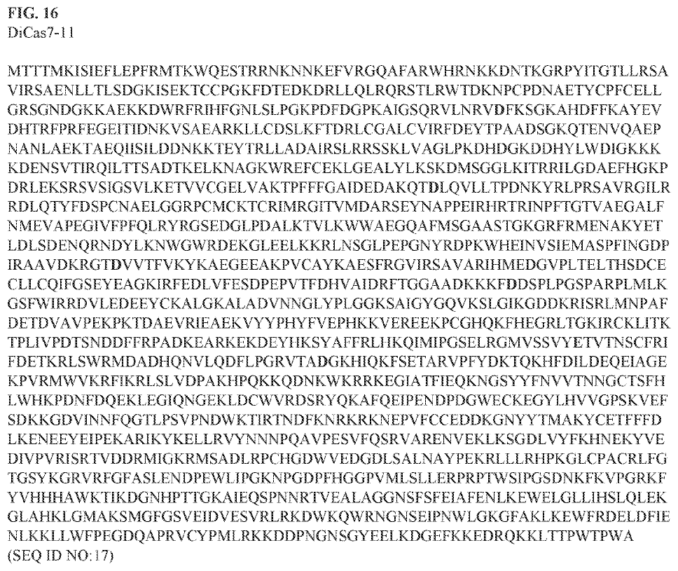

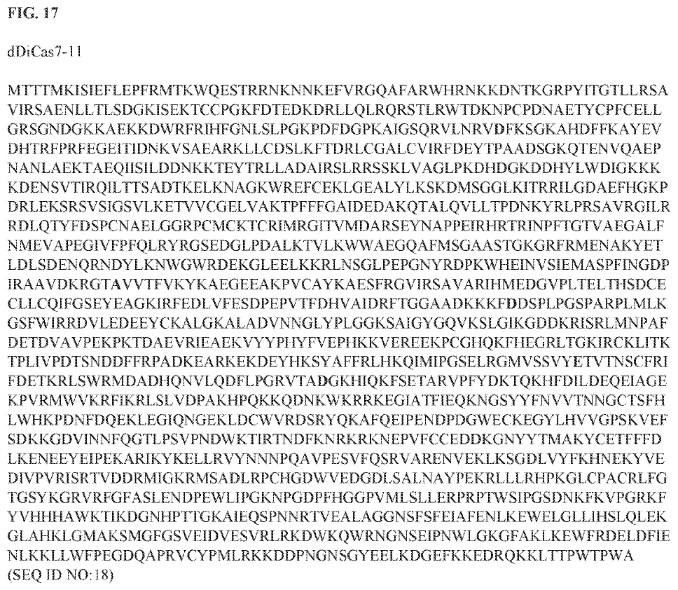

As another example, in some cases, the CRISPR-Cas effector polypeptide is a Cas7-11 polypeptide (e.g., a DiCas7-11 polypeptide). See, e.g., Ozcan et at. (2021) Nature 597:720. In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 16 (DiCas7-11). In some cases, a suitable CRISPR-Cas effector polypeptide comprises an amino acid sequence having at least 50%, at least 60%, at least 70%, at least 80%, at least 85%, at least 90%, at least 95%, at least 98%, at least 99%, or 100%, amino acid sequence identity to the amino acid sequence depicted in FIG. 17 (dDiCas7-11). In some cases, the Cas7-11 polypeptide is a variant that exhibits reduced catalytic activity compared to a wild-type Cas7-11 effector polypeptide. For example, in some cases, the variant Cas7-11 polypeptide comprises a substitution of one or more of D177, D429, D654, D758, E959, and D998 (where the amino acid numbering is as set forth in FIG. 16). D177, D429, D654, D758, E959, and D998 are in bold in FIG. 16. For example, as shown in FIG. 17, a variant Cas7-11 can have an Asp at position 177, an Ala at position 429, an Ala at position 654, an Asp at position 758, a Glu at position E959, and an Asp at position 998. In some cases, a variant Cas7-11 comprises a D429A substitution.

### Fusion CRISPR-Cas Effector Polypeptides

In some cases, a suitable CRISPR-Cas effector polypeptide is a fusion polypeptide comprising: (i) a CRISPR-Cas effector polypeptide; and (ii) one or more heterologous polypeptides (also referred to herein as “fusion partners”). Such polypeptides may be referred to herein as “fusion CRISPR-Cas effector polypeptides.” The fusion partner can be at the N-terminus of the CRISPR-Cas effector polypeptide. The fusion partner can be at the C-terminus of the CRISPR-Cas effector polypeptide.

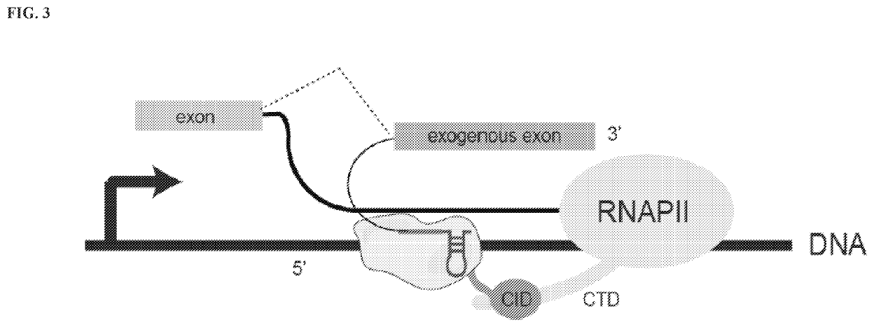

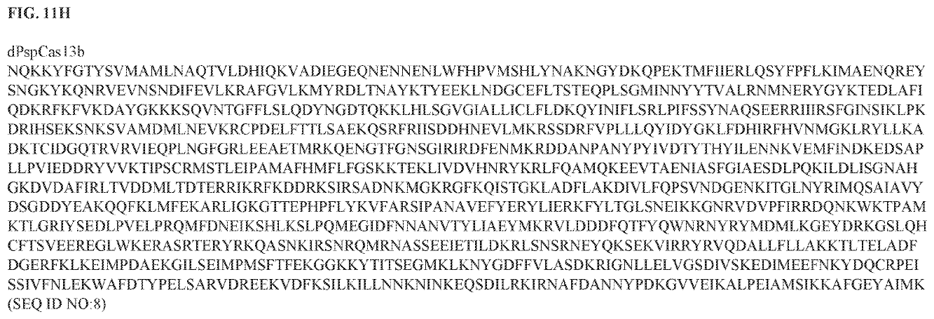

As one example, in some cases, a fusion CRISPR-Cas effector polypeptide comprises: (i) a CRISPR-Cas effector polypeptide; (ii) a C-terminal interacting domain (CID) polypeptide. As another example, in some cases, a fusion CRISPR-Cas effector polypeptide comprises: (i) a CRISPR-Cas effector polypeptide; (ii) a C-terminal interacting domain (CID) polypeptide; and (iii) a double-stranded RNA Binding Domain (ds-RBD). As another example, in some cases, a fusion CRISPR-Cas effector polypeptide comprises: (i) a CRISPR-Cas effector polypeptide; (ii) a CID polypeptide; and (iii) a protein that is associated with the C-terminal domain (CTD) of RNA polymerase II. A CID domain interacts with the C-terminal domain (CTD) of RNA polymerase II (RNAPII), and allows the CRISPR-Cas effector polypeptide to associate with RNAPII as it transcribes, leading to faster localization of the donor RNA to the target RNA. FIG. 3 provides a schematic illustration of use of such a fusion CRISPR-Cas effector polypeptide. In some cases, the CTD polypeptide includes up to 52 repeats of the amino acid sequence YSPTSPS (SEQ ID NO:63). See, e.g., Barron-Casella and Corden (1992) J. Mol. Evol. 35:405; and Winterzerith et al. (1992) Nucl. Acids Res. 20:910; and NP_000928. As an example, a CTD can comprise an amino acid sequence having at least 85%, at least 90%, at least 95%, at least 98%, at least 99%, or 100%, amino acid sequence identity to the CTD amino acid sequence depicted in FIG. 12; and can have a length of from about 300 amino acids to about 375 amino acids (e.g., from 300 amino acids to 350 amino acids, from 350 amino acids to 370 amino acids, or from 370 amino acids to 375 amino acids; e.g., about 368 amino acids in length).

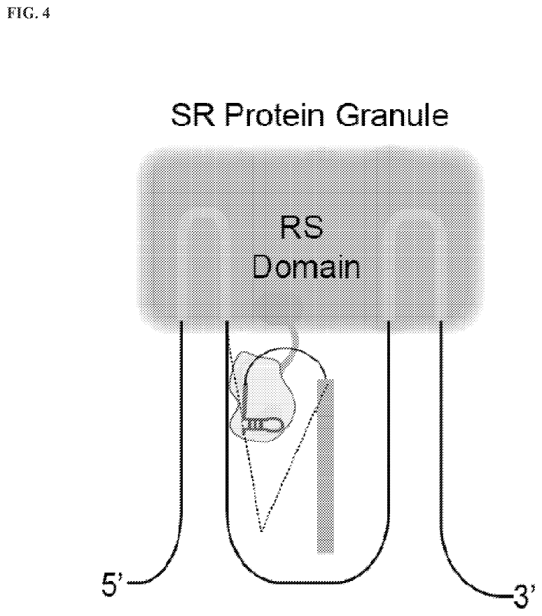

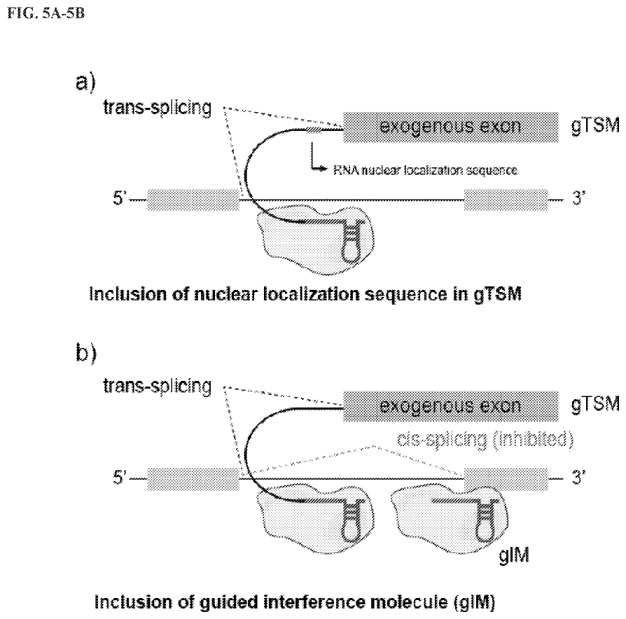

As another example, a fusion CRISPR-Cas effector polypeptide comprises: (i) a CRISPR-Cas effector polypeptide; and (ii) an Arg/Ser-rich (RS) domain polypeptide from a Serine-arginine (SG) polypeptide. Such a fusion partner can increase localization to SR protein granules, which are located proximal to the splicing reaction. See, e.g., Graveley and Maniatis (1998) Molecular Cell 1:765. FIG. 4 provides a schematic illustration of use of such a fusion CRISPR-Cas effector polypeptide.

As another example, at least one of the one or more heterologous polypeptides can comprise a nuclear localization sequence (NLS). In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like). In some cases, the fusion partner is a reverse transcriptase. In some cases, the fusion partner is a base editor. In some cases, the fusion partner is a deaminase.

Other suitable fusion partners include, e.g., polypeptides that can cleave RNA (e.g., a PIN endonuclease, an NYN domain, an SMR domain from SOT1, or an RNase domain from a Staphylococcal nuclease); polypeptides that can affect RNA stability (e.g., tristetraprolin (TTP) or domains from UPF1, EXOSC5, and STAU1); polypeptides that can modify a nucleotide or ribonucleotide (e.g., a cytidine deaminase, PPR protein, adenosine deaminase, an adenosine deaminase acting on RNA (ADAR) family protein, or an APOBEC family protein); polypeptides that can activate translation (e.g., eIF4E and other translation initiation factors, a domain of the yeast poly(A)-binding protein or GLD2), those that can repress translation (e.g., Pumilio or FBF PUF proteins, deadenylases, CAF1, Argonaute proteins); polypeptides that can methylate RNA (e.g., domains from m6A methyltransferase factors such as METTL14, METTL3, or WTAP); polypeptides that can demethylate RNA (e.g., human alkylation repair homolog 5 or Alkbh5); polypeptides that can affect splicing (e.g., the RS-rich domain of SRSF1, the Gly-rich domain of hnRNP A1, the alanine-rich motif of RBM4, or the proline-rich motif of DAZAP1); polypeptides that can enable affinity purification or immunoprecipitation (e.g., FLAG, hemagglutinin (HA), biotin, or HALO tags); and polypeptides that can enable proximity-based protein labeling and identification (e.g., a biotin ligase (such as BirA) or a peroxidase (such as APEX2) in order to biotinylate proteins that interact with the target RNA). Suitable heterologous polypeptides include splicing factors (e.g., RS domains); protein translation components (e.g., translation initiation, elongation, and/or release factors; e.g., eIF4G); RNA methylases; RNA editing enzymes (e.g., RNA deaminases, e.g., ADAR polypeptides, including A to I and/or C to U editing enzymes); helicases; RNA-binding proteins; and the like. In some cases, a heterologous polypeptide (a fusion partner) is a transglycosylase. In some cases, a heterologous polypeptide (a fusion partner) is capable of catalyzing aminoacylation (attachment of an amino acid to a tRNA). In some cases, a heterologous polypeptide (a fusion partner) is capable of catalyzing pseudouridylation (conversion of a uridine to a pseudouridine in an RNA). In some cases, a heterologous polypeptide (a fusion partner) provides for subcellular localization, i.e., the heterologous polypeptide contains a subcellular localization sequence (e.g., a nuclear localization signal (NLS) for targeting to the nucleus; a sequence to keep the fusion protein out of the nucleus, e.g., a nuclear export sequence (NES); a sequence to keep the fusion protein retained in the cytoplasm; a mitochondrial localization signal for targeting to the mitochondria; a chloroplast localization signal for targeting to a chloroplast; an endoplasmic reticulum (ER) retention signal; and the like).

### Hybrid RNA Comprising a CRISPR-Cas Guide RNA and a Donor RNA

In some cases, a hybrid RNA molecule of the present disclosure comprises, in a single RNA molecule: a) a CRISPR-Cas guide RNA; and b) a donor RNA. A CRISPR-Cas guide RNA (referred to herein as a “guide RNA” or “gRNA”) comprises: (i) a targeting region comprising a nucleotide sequence that hybridizes to a target sequence in a target RNA; and (ii) a binding region comprising a nucleotide sequence that binds to (and may also activate) a CRISPR-Cas effector polypeptide. In some cases, the binding region comprises a direct repeat.

In some cases, the binding region (also referred to herein as the “protein-binding region” or “constant region”) is 5′ of the targeting region. The targeting region comprises a guide sequence (also referred to as a “spacer” sequence) that hybridizes to a target sequence in a target RNA.

In some cases, the guide sequence is 16-30 nucleotides (nt) in length (e.g., 16-26, 16-24, 16-22, 16-20, 16-18, 17-26, 17-24, 17-22, 17-20, 17-18, 18-26, 18-24, 18-22, or 18-30 nt in length). In some cases, the guide sequence is 18-24 nucleotides (nt) in length. In some cases, the guide sequence is at least 16 nt long (e.g., at least 18, 20, or 22 nt long). In some cases, the guide sequence is at least 17 nt long. In some cases, the guide sequence is at least 18 nt long. In some cases, the guide sequence is at least 20 nt long. In some cases, the guide sequence is 16-28 nt long.

In some cases, the guide sequence has 80% or more (e.g., 85% or more, 90% or more, 95% or more, or 100% complementarity) with the target sequence of the single stranded target RNA. In some cases, the guide sequence is 100% complementary to the target sequence of the single stranded target RNA. In some cases, the guide sequence has 1, 2, 3, or 4 mismatches with a corresponding stretch of nucleotides in the target sequence. For example, where the guide sequence has a length of 20 nt, the guide sequence can have 1, 2, 3, or 4 mismatches with a corresponding stretch of 20 nt in the target sequence.

The constant region of the guide RNA binds to a CRISPR-Cas effector polypeptide. Such constant region sequences are known in the art. For example, the constant region suitable for use with a Listeria seeligeri Cas13a polypeptide can comprise the following nucleotide sequence: GACUACCUCUAUAUGAAAGAGGACUAAAAC (SEQ ID NO:38). As another example, the constant region suitable for use with a Leptotrichia buccalis Cas13A polypeptide can comprise the following nucleotide sequence: GACCACCCCAAAAAUGAAGGGGACUAAAACA (SEQ ID NO:39). As another example, a guide RNA can comprise a direct repeat (CRISPR-Cas binding region) comprising the sequence: CAAGTAAACCCCTACCAACTGGTCGGGGTTTGAAAC (SEQ ID NO:40), e.g., where the CRISPR-Cas effector polypeptide is an RfxCas13d polypeptide (see, e.g., FIG. 11E and FIG. 11F, respectively, for dead RfxCas13d (dRfxCas13d) and RfxCas13d amino acid sequences). As another example, a guide RNA can comprise a direct repeat (CRISPR-Cas binding region) comprising the sequence: GTTGTGGAAGGTCCAGTTTTGAGGGGCTATTACAAC (SEQ ID NO:41), e.g., where the CRISPR-Cas effector polypeptide is a PspCas13b polypeptide or a dPspCas13b polypeptide (see, e.g., FIG. 11G and FIG. 11H, respectively, for PspCas13b and dead PspCas13b (dPspCas13b) amino acid sequences). As another example, a guide RNA can comprise a direct repeat (CRISPR-Cas binding region) comprising the sequence: GTTGATGTCACGGAAC (SEQ ID NO:42), e.g., where the CRISPR-Cas effector polypeptide is a Cas7-11 polypeptide or a dCas7-11 polypeptide (see, e.g., FIG. 16 for the amino acid sequence of a Cas7-11 polypeptide; and FIG. 17 for the amino acid sequence of a dead Cas7-11 (dCas7-11) polypeptide). As another example, a guide RNA can comprise a direct repeat (CRISPR-Cas binding region) comprising the sequence: CAACTACAACCCCGTAAAAATACGGGGTTCTGAAAC (SEQ ID NO:43), where the CRISPR-Cas effector polypeptide is a DjCas13d polypeptide. As another example, a guide RNA can comprise a direct repeat (CRISPR-Cas binding region) comprising the sequence: GACCAACACCTCTGCAAAACTGCAGGGGTCTAAAAC (SEQ ID NO:44), where the CRISPR-Cas effector polypeptide is an AdmCas13D polypeptide.

The following description relates to Cas13a guide RNAs. However, the description can be generally applied to guide RNAs.

In some cases, the Cas13a guide RNA includes a double stranded RNA duplex (dsRNA duplex).

In some cases, a Cas13a guide RNA includes a dsRNA duplex that is 4 base pairs (bp) in length. In some cases, a Cas13a guide RNA includes a dsRNA duplex with a length of from 2 to 12 bp (e.g., from 2 to 10 bp, 2 to 8 bp, 2 to 6 bp, 2 to 5 bp, 2 to 4 bp, 3 to 12 bp, 3 to 10 bp, 3 to 8 bp, 3 to 6 bp, 3 to 5 bp, 3 to 4 bp, 4 to 12 bp, 4 to 10 bp, 4 to 8 bp, 4 to 6 bp, or 4 to 5 bp). In some cases, a Cas13a guide RNA includes a dsRNA duplex that is 2 or more bp in length (e.g., 3 or more, 4 or more, 5 or more, 6 or more, or 7 or more bp in length). In some cases, a Cas13a guide RNA includes a dsRNA duplex that is longer than the dsRNA duplex of a corresponding wild type Cas13a guide RNA.

In some cases, the region of a Cas13a guide RNA that is 5′ of the guide sequence is 15 or more nucleotides (nt) in length (e.g., 18 or more, 20 or more, 21 or more, 22 or more, 23 or more, 24 or more, 25 or more, 26 or more, 27 or more, 28 or more, 29 or more, 30 or more, 31 or more nt, 32 or more, 33 or more, 34 or more, or 35 or more nt in length). In some cases, the region of a Cas13a guide RNA that is 5′ of the guide sequence is 29 or more nt in length.

In some cases, the region of a Cas13a guide RNA that is 5′ of the guide sequence has a length in a range of from 12 to 100 nt (e.g., from 12 to 90, 12 to 80, 12 to 70, 12 to 60, 12 to 50, 12 to 40, 15 to 100, 15 to 90, 15 to 80, 15 to 70, 15 to 60, 15 to 50, 15 to 40, 20 to 100, 20 to 90, 20 to 80, 20 to 70, 20 to 60, 20 to 50, 20 to 40, 25 to 100, 25 to 90, 25 to 80, 25 to 70, 25 to 60, 25 to 50, 25 to 40, 28 to 100, 28 to 90, 28 to 80, 28 to 70, 28 to 60, 28 to 50, 28 to 40, 29 to 100, 29 to 90, 29 to 80, 29 to 70, 29 to 60, 29 to 50, or 29 to 40 nt). In some cases, the region of a Cas13a guide RNA that is 5′ of the guide sequence has a length in a range of from 28 to 100 nt. In some cases, the region of a Cas13a guide RNA that is 5′ of the guide sequence has a length in a range of from 28 to 40 nt.

In some cases, the region of the Cas13a guide RNA that is 5′ of the guide sequence is truncated relative to (shorter than) the corresponding region of a corresponding wild type Cas13a guide RNA. For example, a mature wild-type Cas13a guide RNA can include a region 5′ of the guide sequence that is 30 nucleotides (nt) in length, and a truncated Cas13a guide RNA (relative to the mature wild-type Cas13a guide RNA) can therefore have a region 5′ of the guide sequence that is less than 30 nt in length (e.g., less than 29, 28, 27, 26, 25, 22, or 20 nt in length). In some cases, a truncated Cas13a guide RNA includes a region 5′ of the guide sequence that has a length in a range of from 12 to 29 nt (e.g., from 12 to 28, 12 to 27, 12 to 26, 12 to 25, 12 to 22, 12 to 20, 12 to 18 nt). In some cases, the truncated Cas13a guide RNA is truncated by one or more nt (e.g., 2 or more, 3 or more, 4 or more, 5 or more, or 10 or more nt), e.g., relative to a corresponding wild type Cas13a guide).

In some cases, the region of the Cas13a guide RNA that is 5′ of the guide sequence is extended relative to (longer than) the corresponding region of a corresponding wild type Cas13a guide RNA. For example, a mature wild-type Cas13a guide RNA can include a region 5′ of the guide sequence that is 30 nucleotides (nt) in length, and an extended Cas13a guide RNA (relative to the mature wild-type Cas13a guide RNA) can therefore have a region 5′ of the guide sequence that is longer than 30 nt (e.g., longer than 31, longer than 32, longer than 33, longer than 34, or longer than 35 nt). In some cases, an extended Cas13a guide RNA includes a region 5′ of the guide sequence that has a length in a range of from 30 to 100 nt (e.g., from 30 to 90, 30 to 80, 30 to 70, 30 to 60, 30 to 50, or 30 to 40 nt). In some cases, the extended Cas13a guide RNA includes a region 5′ of the guide sequence that is extended (e.g., relative to the corresponding region of a corresponding wild type Cas13a guide RNA) by one or more nt (e.g., 2 or more, 3 or more, 4 or more, 5 or more, or 10 or more nt).

In some cases, a Cas13a guide RNA is 30 or more nucleotides (nt) in length (e.g., 34 or more, 40 or more, 45 or more, 50 or more, 55 or more, 60 or more, 65 or more, 70 or more, or 80 or more nt in length). In some cases, the Cas13a guide RNA is 35 or more nt in length.

In some cases, a subject Cas13a guide RNA has a length in a range of from 30 to 120 nt (e.g., from 30 to 110, 30 to 100, 30 to 90, 30 to 80, 30 to 70, 30 to 60, 35 to 120, 35 to 110, 35 to 100, 35 to 90, 35 to 80, 35 to 70, 35 to 60, 40 to 120, 40 to 110, 40 to 100, 40 to 90, 40 to 80, 40 to 70, 40 to 60, 50 to 120, 50 to 110, 50 to 100, 50 to 90, 50 to 80, or 50 to 70 nt). In some cases, the Cas13a guide RNA has a length in a range of from 33 to 80 nt. In some cases, the Cas13a guide RNA has a length in a range of from 35 to 60 nt.

In some cases, a subject Cas13a guide RNA is truncated relative to (shorter than) a corresponding wild type Cas13a guide RNA. For example, a mature wild-type Cas13a guide RNA can be 50 nucleotides (nt) in length, and a truncated Cas13a guide RNA (relative to the Lse Cas13a guide RNA) can therefore in some cases be less than 50 nt in length (e.g., less than 49, 48, 47, 46, 45, 42, or 40 nt in length). In some cases, a truncated Cas13a guide RNA has a length in a range of from 30 to 49 nt (e.g., from 30 to 48, 30 to 47, 30 to 46, 30 to 45, 30 to 42, 30 to 40, 35 to 49, 35 to 48, 35 to 47, 35 to 46, 35 to 45, 35 to 42, or 35 to 40 nt). In some cases, the truncated Cas13a guide RNA is truncated by one or more nt (e.g., 2 or more, 3 or more, 4 or more, 5 or more, or 10 or more nt), e.g., relative to a corresponding wild type Cas13a guide).

In some cases, a subject Cas13a guide RNA is extended relative to (longer than) a corresponding wild type Cas13a guide RNA. For example, a mature wild-type Cas13a guide RNA can be 50 nucleotides (nt) in length, and an extended Cas13a guide RNA (relative to the mature wild-type Cas13a guide RNA) can therefore in some cases be longer than 50 nt (e.g., longer than 51, longer than 52, longer than 53, longer than 54, or longer than 55 nt). In some cases, an extended Cas13a guide RNA has a length in a range of from 51 to 100 nt (e.g., from 51 to 90, 51 to 80, 51 to 70, 51 to 60, 53 to 100, 53 to 90, 53 to 80, 53 to 70, 53 to 60, 55 to 100, 55 to 90, 55 to 80, 55 to 70, or 55 to 60 nt). In some cases, the extended Cas13a guide RNA is extended (e.g., relative to a corresponding wild type Cas13a guide RNA) by one or more nt (e.g., 2 or more, 3 or more, 4 or more, 5 or more, or 10 or more nt).

### Protein-Recruiting Region

As noted above, in some cases, a hybrid RNA molecule of the present disclosure will comprise, in addition to a targeting region and a donor RNA, a protein-recruiting region. In some cases, the protein-recruiting region comprises a nucleotide sequence that binds one or more spliceosome polypeptides. Spliceosome polypeptides include, e.g., U1, U2, U4, U5, and U6.

### Stabilization Region

As noted above, in some cases, a hybrid RNA molecule of the present disclosure will comprise, in addition to a targeting region and a donor RNA, a stabilization region. In some cases, the stabilization region is capable of forming a secondary structure. Examples of such secondary structures include, e.g., pseudoknots, stem-loops, and tetraloops. See, e.g., Shalybkova et al. (2021) RNA 27:907. In some cases, stabilization region comprises a toehold switch. A stabilization region can include a structured RNA motif such as AsCpf1BB, BoxB, a pseudoknot (decoy), a pseudoknot (tEvoPreQ1), fmpknot, mpknot, MS2, PP7, SLBP, TAR, or ThermoPh. In some cases, the stabilization region comprises a pseudoknot, where the pseudoknot is a naturally occurring pseudoknot or a synthetic pseudoknot. The term “pseudoknot,” as used herein, includes, but is not limited to, hairpins, multiloops, kissing loops, coaxial stacking, triplexes, pseudoknot-like structures, a pseudoknotted hairpins and/or a decoy pseudoknotted hairpins or other RNA structural motifs. A hybrid RNA molecule of the present disclosure can include a stabilization region at the 5′ end of the hybrid RNA molecule, at the 3′ end of the hybrid RNA molecule, or at both the 5′ end and the 3′ end of the hybrid RNA molecule.

As one non-limiting example, a stabilization region can comprise the following nucleotide sequence: TTGACGCGGTTCTATCTAGTTACGCGTTAAACCAACTAGAAA (SEQ ID NO:45) (or where the Ts are replaced with Us); where such a sequence is a modified prequeosine riboswitch aptamer (evopreQ1).

As another non-limiting example, a stabilization region can comprise the following nucleotide sequence: GGGTCAGGAGCCCCCCCCCTGAACCCAGGATAACCCTCAAAGTCGGGGGGC (SEQ ID NO:46); e.g., a Moloney murine leukemia virus (MMLV) (mpknot-1 trimmed).

As another non-limiting example, a stabilization region can comprise an mpknot. See, e.g., Nelson et al. (2022) Nat. Biotechnol. 40:402. For example, a stabilization region can comprise the following nucleotide sequence: GTCAGGGTCAGGAGCCCCCCCCCTGAACCCAGGATAACCCTCAAAGTCGGGGGGCAACCC (SEQ ID NO:47). As another example, a stabilization region can comprise the following nucleotide sequence: GGGTCAGGAGCCCCCCCCCTGAACCCAGGAAAACCCTCAAAGTCGGGGGGCAACCC (SEQ ID NO:48). As another example, a stabilization region can comprise the following nucleotide sequence:

(SEQ ID NO: 49)
GGGTCAGGAGCCCCCCCCCTGCACCCAGGAAAACCCTCA
AAGTCGGGGGGCAACCC.

In some cases, a pseudoknot suitable for including in a stabilization region is a tEvoPreQI Pseudoknot comprising the following nucleotide sequence: UAAUUUCUACUAAGUGUAGAU (SEQ ID NO:50). In some cases, a pseudoknot suitable for including in a stabilization region is a pseudoknot EvoPreQI comprising the following nucleotide sequence: TTGACGCGGTTCTATCTAGTTACGCGTTAAACCAACUAGAAA (SEQ ID NO:51) In some cases, a pseudoknot suitable for including in a stabilization region is a pseudoknot comprising the following nucleotide sequence: TAAGTCTCCATAGAATGGAGG (SEQ ID NO:52). In some cases, a pseudoknot suitable for including in a stabilization region is a pseudoknot comprising the following nucleotide sequence: UAAGUCUCCAUAGAAUGGAGG (SEQ ID NO:52).

In some cases, a stabilization region comprises a structured RNA motif, where suitable structured RNA motifs include, but are not limited to, AsCpf1BB, comprising the following nucleotide sequence: TAATTTCTACTCTTGTAGAT (SEQ ID NO:53); BoxB, comprising the following nucleotide sequence: GGGCCCTGAAGAAGGGCCC (SEQ ID NO:54); pseudoknot (decoy), comprising the following nucleotide sequence: TAAGTCTCCATAGAATGGAGG (SEQ ID NO:52); pseudoknot (tEvoPreQI), comprising the following nucleotide sequence: TTGACGCGGTTCTATCTAGTTACGCGTTAAACCAACUAGAAA (SEQ ID NO:51); fmpknot, comprising the following nucleotide sequence: GGAGGTCAGGGTCAGGAGCCCCCCCCTGAACCCAGGATAACCCTCAAAGTCG GGGGGCAACCC (SEQ ID NO:55); mpknot, comprising the following nucleotide sequence: GGGTCAGGAGCCCCCCCCTGAACCCAGGATAACCCTCAAAGTCGGGGGGCA ACCC (SEQ ID NO:56); MS2, comprising the following nucleotide sequence: GGCCAACATGAGGATCACCCATGTCTGCAGGGCC (SEQ ID NO:57); PP7, comprising the following nucleotide sequence: CCGGAGCAGACGATATGGCGTCGCTCCGG (SEQ ID NO:58); SLBP, comprising the following nucleotide sequence: CCAAAGGCTCTTCTCAGAGCCACCCA (SEQ ID NO:59); TAR, comprising the following nucleotide sequence: GGCCAGATCTGAGCCTGGGAGCTCTCTGGCC (SEQ ID NO:60); and ThermoPh, comprising the following nucleotide sequence: ATATAACCTTCACCATTAGGTTCAAATAATGGTAAT (SEQ ID NO:61).

### Nuclear Localization Motif

As noted above, in some cases, a hybrid RNA molecule of the present disclosure will comprise, in addition to a targeting region and a donor RNA, a SINE-derived nuclear RNA Localization (SIRLOIN) motif. See, e.g., Lubelsky and Ulitsky (2018) Nature 555:107. A SIRLOIN motif is a 42-nucleotide motif. In some cases, a SIRLOIN motif comprises the following nucleotide sequence:

(SEQ ID NO: 62)
CGCCTCCCGGGTTCAAGCGATTCTCCTGCCTCAGCCTCCCGA

### RNA Modifications

A hybrid RNA molecule of the present disclosure can include one or more of: a) a modified nucleoside comprising a modified nucleoside base; b) a modified nucleoside comprising a modified sugar; c) a modified internucleoside linkage; and d) a modified backbone linking two or more nucleosides.

A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′O-methyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleoside (also referred to as 2′-O-Methyl RNA) is a naturally occurring modification of RNA found in tRNA and other small RNAs that arises as a post-transcriptional modification. RNA can be directly synthesized so that it contains one or more 2′-O-Methyl nucleosides. Such an RNA is stable with respect to attack by single-stranded ribonucleases.

2′ Fluoro modified nucleosides (e.g., 2′ Fluoro bases) have a fluorine modified ribose which increases binding affinity (Tm) and also confers some relative nuclease resistance when compared to native RNA.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an RNA at any position except the 3′-end.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleoside linkage resistant to nuclease degradation. Phosphorothioate bonds can be introduced between the last 3-5 nucleosides at the 5′- or 3′-end of an RNA molecule to inhibit exonuclease degradation. Including phosphorothioate bonds within an RNA molecule can help reduce attack by endonucleases as well.

In some cases, a subject hybrid RNA molecule has one or more nucleotides that are 2′-O-Methyl modified nucleosides. In some cases, a subject hybrid RNA molecule has one or more 2′ Fluoro modified nucleosides. In some cases, a subject hybrid RNA molecule has one or more LNA bases. In some cases, a subject hybrid RNA molecule has one or more nucleosides that are linked by a phosphorothioate bond (i.e., the subject nucleic acid has one or more phosphorothioate linkages).

### Modified Backbones and Modified Internucleoside Linkages

In some cases, a hybrid RNA molecule of the present disclosure comprises a modified backbone or one or more non-natural internucleoside linkage. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Suitable RNA molecules having inverted polarity comprise a single 3′ to 3′ linkage at the 3′-most internucleoside linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a subject hybrid RNA molecule comprises one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleoside linkage is represented as —O—P(═O)(OH)—O—CH₂—). MMI type internucleoside linkages are disclosed in U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are hybrid RNA molecule having morpholino backbone structures as described in, e.g., U.S. Pat. No. 5,034,506. For example, in some cases, a subject hybrid RNA molecule comprises a 6-membered morpholino ring in place of a ribose ring. In some of these instances, a phosphorodiamidate or other non-phosphodiester internucleoside linkage replaces a phosphodiester linkage.

Suitable modified polynucleoside backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Modified Sugar Moieties

A subject hybrid RNA molecule can also include one or more substituted sugar moieties.

Suitable polynucleotides comprise a sugar substituent group selected from: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH, O(CH₂)ₙOCH₃, O(CH₂)ₙNH₂, O(CH₂)ₙCH₃, O(CH₂)ₙONH₂, and O(CH₂)ₙON((CH₂)˜CH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNA, or a group for improving the pharmacodynamic properties of an RNA, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂₀N(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the RNA, particularly the 3′ position of the sugar on the 3′ terminal nucleoside or in 2′-5′ linked oligonucleosides and the 5′ position of 5′ terminal nucleoside.

### Base Modifications and Substitutions

A subject hybrid RNA molecule may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Nucleic Acids; DNA Molecules; Recombinant Expression Vectors)

The present disclosure provides a DNA molecule comprising a nucleotide sequence encoding a hybrid RNA molecule of the present disclosure. In some cases, the nucleotide sequence is operably linked to transcriptional control element, such as a promoter, e.g., a promoter functional in a eukaryotic cell. In some cases, a DNA molecule of the present disclosure comprises a nucleotide sequence encoding a hybrid RNA molecule and a CRISPR-Cas effector polypeptide or a CRISPR-Cas fusion polypeptide. In some cases, a hybrid RNA molecule and a CRISPR-Cas effector polypeptide (or CRISPR-Cas fusion polypeptide) are encoded on separate DNA molecules. In some cases, a nucleotide sequence encoding a hybrid RNA molecule is codon optimized, e.g., for production in a particular organism.

The present disclosure provides a recombinant expression vector comprising a DNA molecule of the present disclosure, e.g., comprising a nucleotide sequence encoding a hybrid RNA molecule of the present disclosure. The present disclosure provides a recombinant expression vector comprising: a) a first nucleotide sequence encoding a hybrid RNA molecule of the present disclosure; and b) a second nucleotide sequence encoding a CRISPR-Cas effector polypeptide or a CRISPR-Cas fusion polypeptide. The present disclosure provides a first recombinant expression vector comprising a nucleotide sequence encoding a hybrid RNA molecule of the present disclosure and a second recombinant expression vector comprising a nucleotide sequence encoding a CRISPR-Cas effector polypeptide or a CRISPR-Cas fusion polypeptide.

Suitable expression vectors include viral expression vectors (e.g., viral vectors based on vaccinia virus; poliovirus; adenovirus; adeno-associated virus; lentivirus, e.g., a human immunodeficiency virus; a retrovirus; etc.). In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

As noted above, in some cases, a nucleotide sequence encoding a hybrid RNA molecule of the present disclosure is operably linked to a transcriptional control element. Similarly, a nucleotide sequence encoding a CRISPR-Cas effector polypeptide or a CRISPR-Cas fusion polypeptide can be operably linked to a transcriptional control element. The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. In some cases, the transcriptional control element is one that is functional in eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

RNA polymerase III (Pol III) promoters can be used to drive the expression of an RNA. In some cases, a suitable promoter is a Pol III promoter. Non-limiting examples of Pol III promoters include a U6 promoter, an H1 promoter, a 5S promoter, an Adenovirus 2 (Ad2) VAI promoter, a tRNA promoter, and a 7SK promoter. See, for example, Schramm and Hernandez (2002) Genes & Development 16:2593-2620. In some cases, a Pol III promoter is selected from the group consisting of a U6 promoter, an H1 promoter, a 5S promoter, an Adenovirus 2 (Ad2) VAI promoter, a tRNA promoter, and a 7SK promoter. In some cases, a hybrid RNA molecule-encoding nucleotide sequence is operably linked to a promoter selected from the group consisting of a U6 promoter, an H1 promoter, a 5S promoter, an Adenovirus 2 (Ad2) VAI promoter, a tRNA promoter, and a 7SK promoter. In some cases, a hybrid RNA molecule-encoding nucleotide sequence is operably linked to a promoter selected from the group consisting of a U6 promoter, an H1 promoter, a 5S promoter, an Adenovirus 2 (Ad2) VAI promoter, a tRNA promoter, and a 7SK promoter.

### Compositions

The present disclosure provides a composition comprising one or more hybrid RNA molecules of the present disclosure. The present disclosure provides a composition comprising a DNA molecule of the present disclosure. The present disclosure provides a composition comprising a recombinant expression vector of the present disclosure. A composition of the present disclosure can comprise, in addition to a hybrid RNA molecule of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

A composition of the present disclosure can include: a) a hybrid RNA molecule of the present disclosure, or a nucleic acid or recombinant expression vector comprising a nucleotide sequence encoding the hybrid RNA molecule; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, a nuclease inhibitor, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a hybrid RNA molecule of the present disclosure, and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

Formulation suitable for parenteral administration can include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

In some cases, a hybrid RNA molecule is formulated with a lipid, e.g., a lipid such as 1,2-dioleoyl-3-trimethylammonium-propane (DOTAP) or 1,2-ditetradecanoyl-sn-glycero-3-phosphocholine (DMPC). In some cases, a hybrid RNA molecule is present in a liposome.

In some cases, a hybrid RNA molecule is in a particle or associated with a particle. A variety of particles (e.g., nanoparticles) can be used, e.g., sugar-based particles, lipid-based particles, and the like. For example, a lipid nanoparticle can be used, where the lipid nanoparticle comprises one or more lipids such as 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA).

In some cases, a hybrid RNA molecule is formulated with a stable nucleic-acid-lipid particle (SNALP). A SNALP may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)₂₀₀₀)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

In some cases, a hybrid RNA molecule is present in a ribonucleoprotein (RNP) complex with a CRISPR-Cas effector polypeptide or a CRISPR-Cas fusion polypeptide. The present disclosure thus provides an RNP comprising: a) a hybrid RNA molecule of the present disclosure; and b) a CRISPR-Cas effector polypeptide or a CRISPR-Cas fusion polypeptide. In these cases, the hybrid RNA molecule comprises, in addition to a targeting region and a donor RNA, a binding region that binds to the CRISPR-Cas effector polypeptide or CRISPR-Cas fusion polypeptide.

### Methods for Modifying a Target RNA

The present disclosure provides methods for modifying a target RNA, such that a modified RNA is produced. The methods comprise contacting a target RNA with a hybrid RNA molecule of the present disclosure.

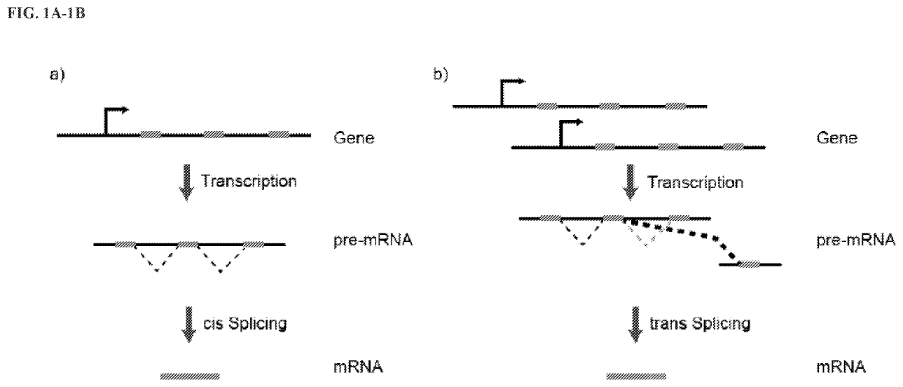

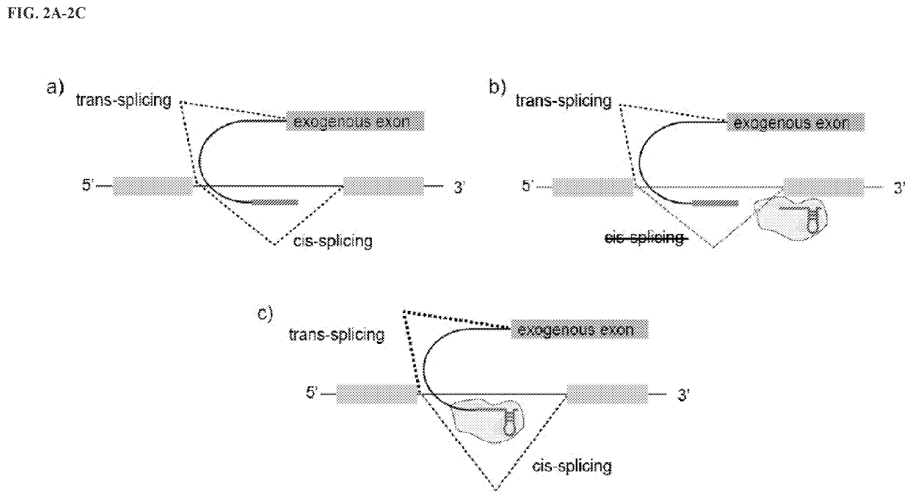

FIG. 1B depicts trans splicing of a pre-mRNA as one non-limiting embodiment. As depicted in FIG. 2A-2C, a method of the present disclosure can include one or more of: a) initiation of trans-splicing via the binding of a trans-splicing RNA molecule (e.g., a hybrid RNA molecule of the present disclosure) to a target RNA; b) inhibition of the competing cis-splicing reaction; and c) enhancement of trans-splicing efficiency. In FIG. 2C, a CRISPR-Cas effector polypeptide binds to the hybrid RNA molecule to increase efficiency of the trans-splicing region and to inhibit cis-splicing.

In some cases, the target RNA is present in a eukaryotic cell. In some cases, the target RNA is present in a eukaryotic cell in vitro. In some cases, the target RNA is present in a eukaryotic cell in vivo. In some cases, the cell is a mammalian cell (e.g., a human cell, a non-human primate cell, a bovine cell, an ovine cell, a porcine cell, an equine cell, a feline cell, a canine cell, a rodent cell, etc.). In some cases, the cell is a stem cell, e.g., a pluripotent cell, a hematopoietic stem cell, a stem cell that gives rise to myocytes, tec. In some cases, the cell is an embryonic germ cell, an induced pluripotent stem cell, a mitotic cell, a meiotic cell, or a somatic cell.

Target RNA that can be modified using a method of the present disclosure include pre-mRNA, microRNA (miRNA), transfer RNA, ribosomal RNA, a circular RNA, a partially spliced RNA, a non-coding RNA, a non-host cell RNA, a regulatory RNA, a coding RNA, a transfer RNA (tRNA), a pre-ribosomal RNA, a ribosomal RNA, a mature RNA with cryptic splice sites, and a long non-coding RNA (lncRNA). In some cases, the target RNA is a pre-mRNA, e.g., an unspliced RNA. In some cases, the target RNA comprises a coding region that encodes an abnormal polypeptide, e.g., a polypeptide that is associated with a disease state. In some cases, the target RNA comprises a defective splice donor and/or a defective splice acceptor.

A hybrid RNA molecule is introduced into a cell (a target cell) comprising a target RNA. A hybrid RNA molecule can be introduced into a target cell using any of a variety of methods. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some cases, a method of the present disclosure comprises administering to an individual in need thereof an effective amount of a hybrid RNA molecule of the present disclosure, or a DNA molecule (or a recombinant expression vector comprising the DNA molecule) comprising a nucleotide sequence encoding a hybrid RNA molecule of the present disclosure. In some cases, a hybrid RNA molecule of the present disclosure is administered to an individual in need thereof, where the hybrid RNA molecule is formulated in a lipid nanoparticle. In some cases, a method of the present disclosure comprises administering to an individual in need thereof an effective amount of a DNA molecule comprising a nucleotide sequence encoding a hybrid RNA molecule of the present disclosure.

In some cases, the DNA molecule is integrated into the genome of a cell in the individual. In some cases, a method of the present disclosure comprises administering to an individual in need thereof an effective amount of a recombinant expression vector comprising a DNA molecule comprising a nucleotide sequence encoding a hybrid RNA molecule of the present disclosure. In some cases, the DNA molecule is integrated into the genome of a cell in the individual. In some cases, the recombinant expression vector, or the nucleotide sequence encoding the hybrid RNA molecule, is integrated into the genome of a cell in the individual. In some cases, the recombinant expression vector is not integrated into the genome of a cell in the individual.

In some cases, a method of the present disclosure for modifying a target RNA comprises contacting the target RNA with a composition comprising: a) a hybrid RNA molecule; and b) a CRISPR-Cas effector polypeptide or a CRISPR-Cas fusion polypeptide. In some cases, the method comprises introducing into a target cell comprising a target RNA: a) a hybrid RNA molecule; and b) a CRISPR-Cas effector polypeptide or a CRISPR-Cas fusion polypeptide. In some cases, the method comprises introducing into a target cell comprising a target RNA: a) a DNA molecule (e.g. a recombinant expression vector) comprising a nucleotide sequence encoding a hybrid RNA molecule; and b) a CRISPR-Cas effector polypeptide or a CRISPR-Cas fusion polypeptide. In some cases, the method comprises introducing into a target cell comprising a target RNA: a) a hybrid RNA molecule; and b) a DNA molecule (e.g. a recombinant expression vector) comprising a nucleotide sequence encoding a CRISPR-Cas effector polypeptide or a CRISPR-Cas fusion polypeptide.

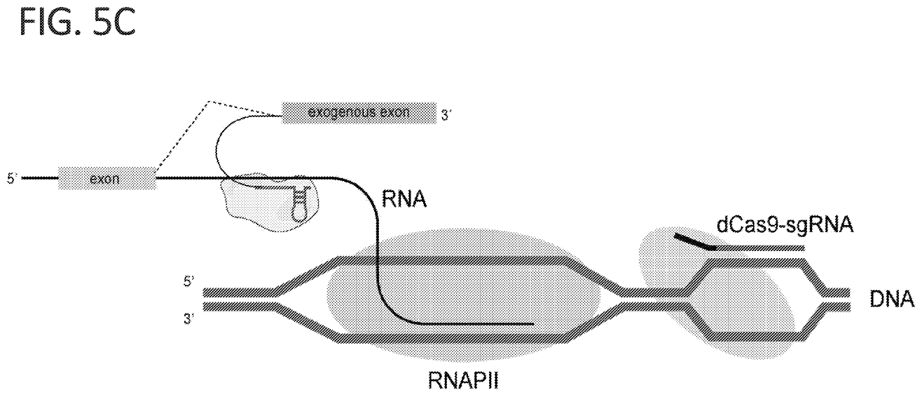

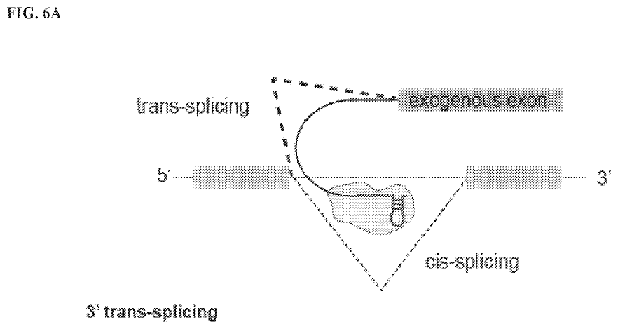

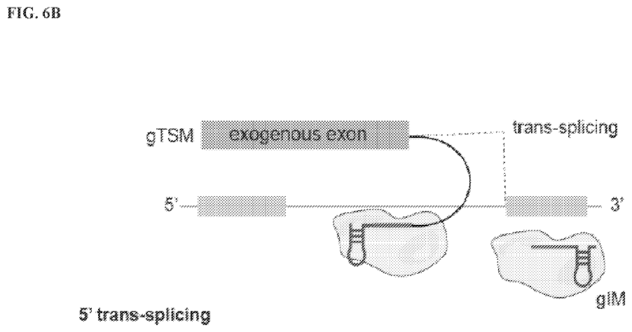

A method of the present disclosure increases trans-splicing, thereby generating a modified RNA that comprises a nucleotide sequence present in the donor RNA of a hybrid RNA molecule of the present disclosure. In some cases, the method further comprises inhibiting cis-splicing. For example, in some cases, the method further comprises contacting a target RNA (e.g., introducing into a target cell comprising a target RNA), with a RNA-targeting CRISPR-Cas guide RNA comprising a targeting region that comprises a nucleotide sequence that is complementary to a second target sequence of the target RNA, wherein the RNA-targeting CRISPR-Cas guide RNA sterically inhibits cis-splicing of the target RNA. Such a molecule is referred to as a “guided interference molecule” (gIM). This is illustrated schematically in FIG. 5A-5B. In some cases, the method further comprises contacting a target DNA (e.g., introducing into a target cell comprising a target DNA) with a DNA-targeting CRISPR-Cas guide RNA comprising a targeting region that comprises a nucleotide sequence that is complementary to a target sequence of the target DNA, wherein the DNA-targeting CRISPR-Cas guide RNA interrupts RNA transcription downstream of the targeted site thereby reducing the number transcripts that are able to splice in cis. This is illustrated schematically in FIG. 5C.

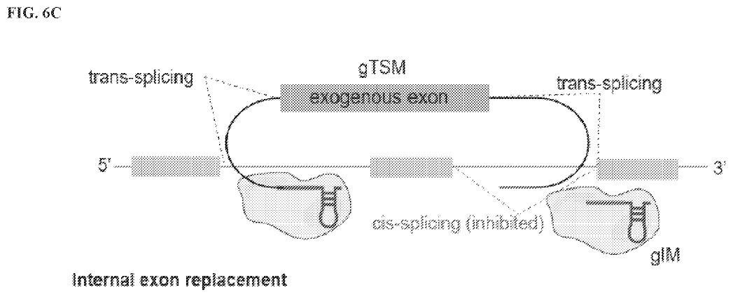

A method for modifying a target RNA according to the present disclosure can be used to insert one or more nucleotides into a target RNA and/or to replace one or more nucleotides in a target RNA. FIG. 6A-6C schematically illustrate various embodiments of modification of a target RNA using trans-splicing.

In some cases, a method of the present disclosure provides for replacement of from 1 nucleotide (nt) to 9000 nt of a target RNA; e.g., a method of the present disclosure provides for replacement of from 1 nt to 10 nt, from 10 nt to 15 nt, from 15 nt to 20 nt, from 20 nt to 25 nt, from 25 nt to 30 nt, from 30 nt to 40 nt, from 40 nt to 50 nt, from 50 nt to 60 nt, from 60 nt to 70 nt, from 70 nt to 80 nt, from 80 nt to 90 nt, from 90 nt to 100 nt, from 100 nt to 250 nt, from 250 nt to 500 nt, from 500 nt to 1000 nt, from 1000 nt to 2000 nt, from 2000 nt to 3000 nt, from 3000 nt to 4000 nt, from 4000 nt to 5000 nt, from 5000 nt to 6000 nt, from 6000 nt to 7000 nt, from 7000 nt to 8000 nt, or from 8000 nt to 9000 nt, of a target RNA. In some cases, a method of the present disclosure provides for replacement of 1 nt, 2 nt, 3 nt, 4 nt 5 nt, 6 nt, 7 nt, 8 nt, 9 nt, or 10 nt, of a target RNA. In some cases, a method of the present disclosure provides for replacement of from 10 nt to 20 nt (e.g., 10, 11, 12, 13, 14, 15, 16, 17, 18, 19, or 20 nt). In some cases, a method of the present disclosure provides for replacement of from 20 nt to 30 nt (e.g., 20, 21, 22, 23, 24, 25, 26, 27, 28, 29, or 30 nt). In some cases, a method of the present disclosure provides for replacement of from 20 nt to 100 nt. In some cases, a method of the present disclosure provides for replacement of from 100 nt to 200 nt. In some cases, a method of the present disclosure provides for replacement of from 100 nt to 250 nt. In some cases, a method of the present disclosure provides for replacement of from 100 nt to 500 nt. In some cases, a method of the present disclosure provides for replacement of from 100 nt to 1000 nt. In some cases, a method of the present disclosure provides for replacement of from 1000 nt to 1500 nt. In some cases, a method of the present disclosure provides for replacement of from 1500 nt to 2000 nt. In some cases, a method of the present disclosure provides for replacement of from 1500 nt to 2500 nt. In some cases, a method of the present disclosure provides for replacement of from 2000 nt to 8000 nt. In some cases, a method of the present disclosure provides for replacement of from 2000 nt to 7000 nt. In some cases, a method of the present disclosure provides for replacement of from 2000 nt to 6000 nt. In some cases, a method of the present disclosure provides for replacement of from 2000 nt to 5000 nt.

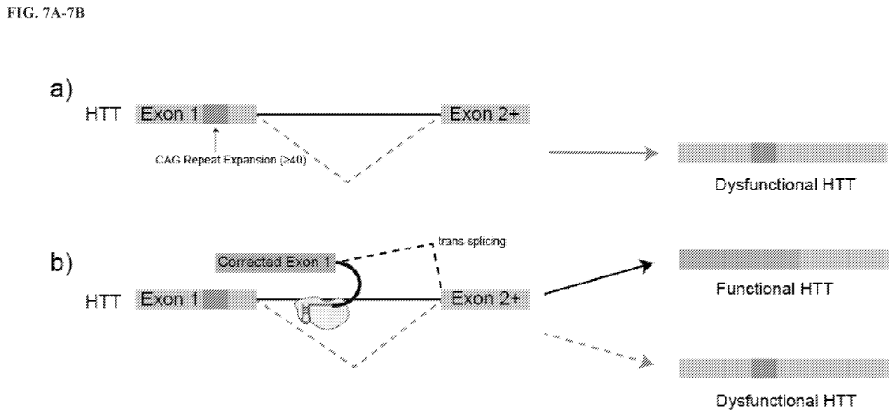

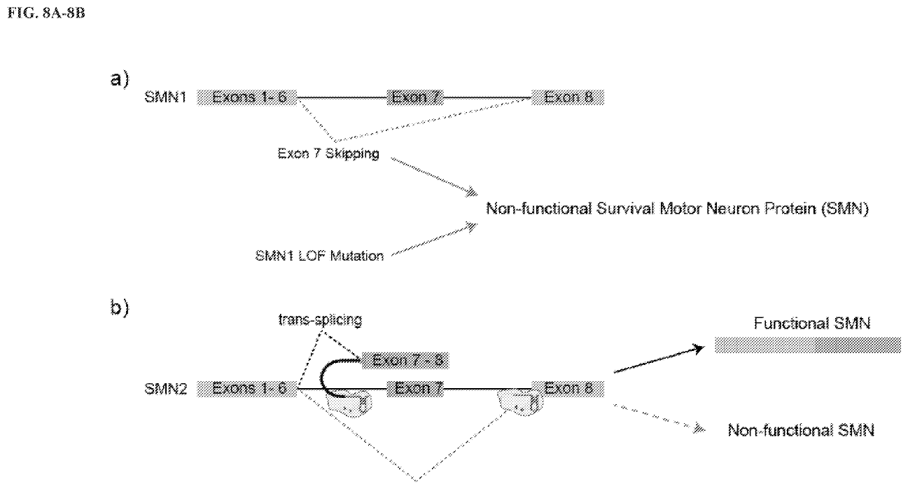

The following are non-limiting examples of use of a method of the present disclosure. These non-limiting examples are depicted schematically in FIG. 7A-7B and in FIG. 8A-8B.

In some cases, a method of the present disclosure modifies a target RNA, where the target RNA is a pre-mRNA encoding a protein comprising a Gln repeat, where the Gln repeat comprises (Gln)n, where n is greater than 30. For example, the pre-mRNA comprises a CAG trinucleotide repeat expansion such that the encoded protein comprises a Gln repeat. Such proteins can give rise to a disease state. For example, a huntingtin protein comprising a poly(Gln) expansion of more than about 30 Gln residues can result in Huntington's disease. As another example, an ataxin polypeptide comprising a poly(Gln) expansion of more than about 30 Gln residues can result in a spinocerebellar ataxia (SCA). An ataxin-1 polypeptide comprising a poly(Gln) expansion of more than about 30 Gln residues can result in SCA type 1 (SCA1). An ataxin-2 polypeptide comprising a poly(Gln) expansion of more than about 30 Gln residues can result in SCA type 2 (SCA2). An ataxin-3 polypeptide comprising a poly(Gln) expansion of more than about 30 Gln residues can result in SCA type 2 (SCA3). As another example, an atrophin-1 polypeptide comprising a poly(Gln) expansion of more than about 30 Gln residues can result in dentatorubral-pallidoluysian atrophy (DRPLA).

In some cases, a method of the present disclosure modifies a target RNA, where the target RNA is a pre-mRNA comprising a GGGGCC hexanucleotide expansion repeat, e.g., where the GGGGCC expansion repeat comprises more than about 25 repeats of the hexanucleotide GGGGCC. Diseases associated with such a hexanucleotide expansion repeat include amyotrophic lateral sclerosis (ALS) and frontotemporal dementia (FTD). Such a pre-mRNA can encode a pathological form of C9orf72, leading to ALS or FTD.

In some cases, a method of the present disclosure modifies a target RNA, where the target RNA is a pre-mRNA comprising a GAA trinucleotide repeat expansion, e.g., where the GAA trinucleotide expansion repeat comprises more than about 25-30 repeats of the trinucleotide GAA. Diseases associated with such trinucleotide repeat expansions include Friedreich's ataxia.

In some cases, a method of the present disclosure provides for reduced production of a disease-associated expansion repeat protein in a cell in an individual. For example, a method of the present disclosure can provide for a reduction of at least 10%, at least 15%, at least 20%, at least 25%, at least 30%, at least 35%, at least 40%, at least 50%, or more than 50%, of a disease-associated expansion repeat protein in a cell in an individual. In some cases, a method of the present disclosure provides for increased production of a normal form (e.g., a form comprising an expansion repeat that does not lead to pathology) of a disease-associated expansion repeat protein in a cell in an individual. For example, in some cases, a method of the present disclosure provides for at a 10%, at least 20%, at least 30%, at least 40%, at least 50%, at least 100% (or 2-fold), at least 2.5-fold, at least 5-fold, at least 10-fold, or more than 10-fold, increased production of a normal form (e.g., a form comprising an expansion repeat that does not lead to pathology) of a disease-associated expansion repeat protein in a cell in an individual.

A method of the present disclosure can be used to treat any trinucleotide or hexanucleotide expansion repeat disease. Examples of diseases that can be treated using a method of the present disclosure include Huntington's Disease, spinal and bulbar muscular atrophy (SBMA), spinocerebellar ataxia (SCA) Type 1, SCA Type 2, SCA Type 3, SCA Type 6, SCA Type 7, SCA Type 8, SCA Type 12, SCA Type 17, Friedreich's ataxia, C90RF72 frontotemporal dementia, Fragile X syndrome, and myotonic dystrophy.

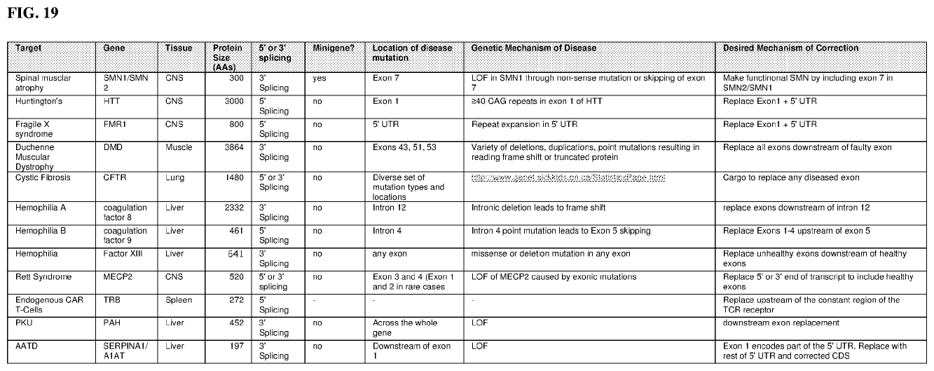

The present disclosure provides methods of treating a disease, the method comprising administering to an individual having such disease a hybrid RNA molecule of the present disclosure, or a DNA molecule comprising a nucleotide sequence encoding the hybrid RNA molecule, or a recombinant expression vector comprising the DNA molecule. A method of the present disclosure can be used to treat any genetic disease, e.g., where an RNA comprises a defective intron, a defective donor splice site, a defective acceptor splice site, a defective exon, and the like. FIG. 19 provides non-limiting examples of diseases that can be treated using a method of the present disclosure.

A target RNA can be present in a cell in vitro (e.g., a cell obtained from an individual in need of treatment), where the cell is modified in vitro using a method of the present disclosure, and the modified cell is introduced into the individual from whom the cell was obtained. A target cell can be present in an individual in vivo. Suitable target cells (where a target cell comprises a target RNA) include, but are not limited to, blood cells, lymphocytes, T cells, hematopoietic stem cells, neuronal stem cells, neurons, glial cells, spleen cells, lung cells (e.g., lung epithelial cells), bone marrow cells, stem cells, and the like.

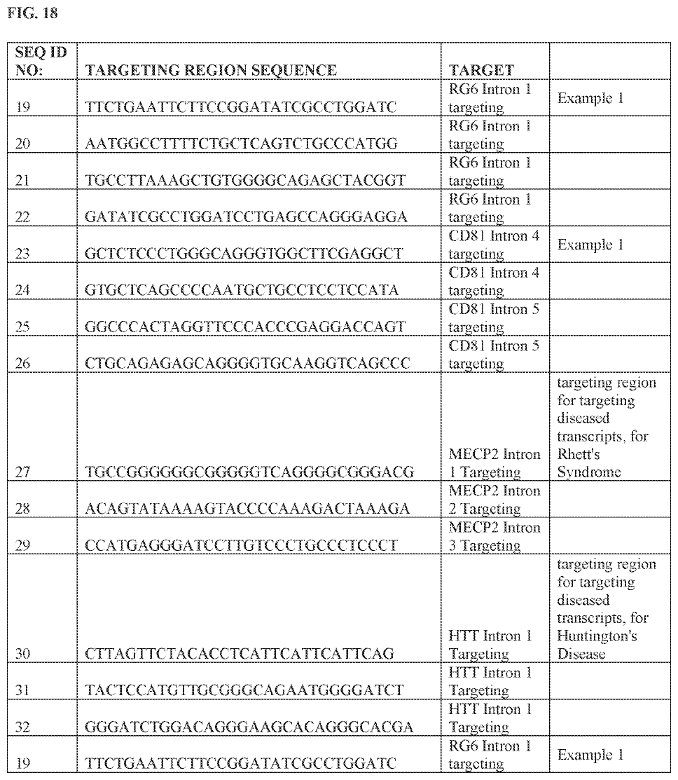

FIG. 18 provides non-limiting examples of targeting region sequences that can be included in a hybrid RNA molecule of the present disclosure.

### Rhett Syndrome

As one example, as set out in FIG. 18, an MECP2 target RNA can be targeted, for use in treating Rhett Syndrome. For example, the target sequence of a MECP2 target RNA can be intron 1, intron 2, or intron 3. A suitable donor sequence can include, e.g., the following sequence:

(SEQ ID NO: 33)
GGAAGAAAAGTCAGAAGACCAGGACCTCCAGGGCCTCAAGGACAA
ACCCCTCAAGTTTAAAAAGGTGAAGAAAGATAAGAAAGAAGAGAA
AGAGGGCAAGCATGAGCCCGTGCAGCCATCAGCCCACCACTCTGC
TGAGCCCGCAGAGGCAGGCAAAGCAGAGACATCAGAAGGGTCAGG
CTCCGCCCCGGCTGTGCCGGAAGCTTCTGCCTCCCCCAAACAGCG
GCGCTCCATCATCCGTGACCGGGGACCCATGTATGATGACCCCAC
CCTGCCTGAAGGCTGGACACGGAAGCTTAAGCAAAGGAAATCTGG
CCGCTCTGCTGGGAAGTATGATGTGTATTTGATCAA.

### Huntington's Disease

As another example, as set out in FIG. 18, a huntingtin-encoding target RNA (HTT target RNA) can be targeted, in connection with treating Huntington's disease. For example, the target sequence of an HTT target RNA can be intron 1. A suitable donor sequence can include, e.g., the following sequence:

(SEQ ID NO: 34)
GCTGCCGGGACGGGTCCAAGATGGACGGCCGCTCAGGTTCTGCTT
TTACCTGCGGCCCAGAGCCCCATTCATTGCCCCGGTGCTGAGCGG
CGCCGCGAGTCGGCCCGAGGCCTCCGGGGACTGCCGTGCCGGGCG
GGAGACCGCCATGGCGACCCTGGAAAAGCTGATGAAGGCCTTCGA
GTCCCTCAAGTCCTTCCAGCAGCAGCAGCAGCAGCAGCAGCAGCA
GCAGCAGCAGCAGCAGCAGCAGCAGCAGCAGCAGCAACAGCCGCC
ACCGCCGCCGCCGCCGCCGCCGCCTCCTCAGCTTCCTCAGCCGCC
GCCGCAGGCACAGCCGCTGCTGCCTCAGCCGCAGCCGCCCCCGCC
GCCGCCCCCGCCGCCACCCGGCCCGGCTGTGGCTGAGGAGCCGCT
GCACCGACC.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects.

This is intended to provide support for all such combinations of aspects and is not limited to combinations of aspects explicitly provided below:

Aspect 1. A hybrid RNA molecule comprising
a) a targeting region that comprises a nucleotide sequence that is complementary to a target sequence of a target RNA; and
b) a donor sequence to said targeting region.

Aspect 2. The hybrid RNA molecule of aspect 1, further comprising one or more of:
a) a binding region that binds to an RNA binding or modifying polypeptide;
b) a protein-recruiting region;
c) a stabilization region.

Aspect 3. The hybrid RNA molecule of aspect 2, wherein the hybrid RNA molecule comprises a binding region that binds to an RNA binding or modifying polypeptide, and wherein the RNA binding or modifying polypeptide is an RNA-targeting CRISPR-Cas effector polypeptide.

Aspect 4. The hybrid RNA molecule of aspect 3, wherein the RNA-targeting CRISPR-Cas effector polypeptide is a Type II, a Type III, or a Type VI CRISPR-Cas effector polypeptide.

Aspect 5. The hybrid RNA molecule of aspect 4, wherein the Type II CRISPR-Cas effector polypeptide is a Cas9 polypeptide.

Aspect 6. The hybrid RNA molecule of aspect 4, wherein the Type VI CRISPR-Cas effector polypeptide is a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide.

Aspect 7. The hybrid molecule of aspect 4, wherein the Type III RNA-targeting CRISPR-Cas effector polypeptide is a Cas7-11 polypeptide.

Aspect 8. The hybrid RNA molecule of aspect 2, wherein the hybrid RNA molecule comprises a protein-recruiting region.

Aspect 9. The hybrid RNA molecule of aspect 8, wherein the protein-recruiting region comprises a nucleotide sequence that binds one or more spliceosome polypeptides.

Aspect 10. The hybrid RNA molecule of aspect 2, wherein the hybrid RNA molecule comprises a stabilization region.

Aspect 11. The hybrid RNA molecule of aspect 10, wherein the stabilization region is capable of forming a secondary structure.

Aspect 12. The hybrid RNA molecule of aspect 11, wherein the secondary structure comprises a pseudoknot, a stem-loop, or a tetraloop.

Aspect 13. The hybrid RNA molecule of any one of aspects 1-12, wherein the RNA donor comprises one or more of:
a) a coding region comprising a nucleotide sequence encoding all or a portion of a polypeptide;
b) an untranslated region (UTR);
c) a bar code;
d) an internal ribosomal entry site (IRES);
c) a poly(pyrimidine) tract;
f) an RNA response element;
g) an intron; and
h) an aptamer.

Aspect 14. The hybrid RNA molecule of any one of aspects 1-13, further comprising a splice function region comprising one or more of:
a) a splice modifying sequence;
b) a splice exclusion sequence;
c) an intron;
d) a branch point;
e) an intronic splice enhancer;
f) an intronic splice silencer; and
g) a poly(pyrimidine tract).

Aspect 15. The hybrid DNA molecule of aspect 14, wherein the splice modifying sequence comprises a splice acceptor, a splice donor, a branchpoint, or a polypyrimidine tract.

Aspect 16. The hybrid RNA molecule of aspect 13, wherein the RNA donor comprises a nucleotide sequence encoding all or a portion of a polypeptide.

Aspect 17. The hybrid RNA molecule of aspect 16, wherein the RNA donor comprises a nucleotide sequence encoding a portion of a non-pathological form of a disease-associated polypeptide, wherein the disease-associated polypeptide comprises an amino acid expansion.

Aspect 18. The hybrid RNA molecule of aspect 17, wherein the disease-associated polypeptide comprises a poly(Gln) repeat region, and wherein the non-pathological form of the polypeptide comprises a (Gln)x repeat region, wherein x is less than the number of repeats associated with a disease.

Aspect 19. The hybrid RNA molecule of aspect 17, wherein the disease-associated polypeptide comprises a poly(Glu) repeat region, and wherein the non-pathological form of the polypeptide comprises a (Glu)x repeat region, wherein x is less than the number of repeats associated with a disease.

Aspect 20. The hybrid RNA molecule of aspect 13, wherein the RNA donor comprises an intron comprising a non-pathological number of repeats of GAA.

Aspect 21. The hybrid RNA molecule of aspect 13, wherein the RNA donor comprises a 5′-UTR comprising a non-pathological number of repeats of CGG, CCG, or GGC.

Aspect 22. The hybrid RNA molecule of aspect 13, wherein the RNA donor comprises a 3′-UTR comprising a non-pathological number of repeats of CTG.

Aspect 23. The hybrid RNA molecule of any one of aspects 1-22, comprising a SINE-derived nuclear RNA Localization (SIRLOIN) motif.

Aspect 24. The hybrid RNA molecule of any one of aspects 1-23, further comprising a second targeting region that comprises a nucleotide sequence that is complementary to a second target sequence of the target RNA and that interferes with cis splicing of the target RNA.

Aspect 25. The hybrid RNA molecule of any one of aspects 1-24, wherein the RNA donor has a length of from 2 nucleotides to 10,000 nucleotides.

Aspect 26. A DNA molecule comprising a nucleotide sequence encoding the hybrid RNA molecule of any one of aspects 1-25.

Aspect 27. The DNA molecule of aspect 26, wherein the nucleotide sequence is operably linked to a transcriptional control element.

Aspect 28. The DNA molecule of aspect 27, wherein the transcriptional control element comprises a promoter, and wherein the promoter is one or more of: a constitutive promoter, an inducible promoter, a cell type-specific promoter, and a tissue-specific promoter.

Aspect 29. A recombinant expression vector comprising the DNA molecule of any one of aspects 26-28.

Aspect 30. A composition comprising:
a) the hybrid RNA molecule of any one of aspects 1-25; and
b) one or more of a nanoparticle, a lipid, a buffer, and a nuclease inhibitor.

Aspect 31. A composition comprising:
a1) an RNA-targeting CRISPR-Cas effector polypeptide; and
b1) a hybrid RNA molecule of any one of aspects 1-25; or
a2) a fusion polypeptide comprising:
i) an RNA-targeting CRISPR-Cas effector polypeptide; and
ii) one or more heterologous polypeptides; and
b2) a hybrid RNA molecule of any one of aspects 1-25.

Aspect 32. The composition of aspect 31, wherein the RNA-targeting CRISPR-Cas effector polypeptide is a Type II, a Type III, or a Type VI CRISPR-Cas effector polypeptide.

Aspect 33. The composition of aspect 32, wherein the Type II CRISPR-Cas effector polypeptide is a Cas9 polypeptide.

Aspect 34. The composition of aspect 32, wherein the Type III CRISPR-Cas effector polypeptide is a Cas7-11 polypeptide.

Aspect 35. The composition of aspect 32, wherein the Type VI CRISPR-Cas effector polypeptide is a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide.

Aspect 36. The composition of aspect 31, wherein the composition comprises:
a) a fusion polypeptide comprising:
i) an RNA-targeting CRISPR-Cas effector polypeptide; and
ii) one or more heterologous polypeptides; and
b) a hybrid RNA molecule of any one of aspects 1-25.

Aspect 37. The composition of aspect 36, wherein at least one of the one or more heterologous polypeptides comprises an RNA Polymerase II-binding polypeptide.

Aspect 38. The composition of aspect 37, wherein the RNA Polymerase II-binding polypeptide comprises a C-terminal interacting domain (CID).

Aspect 39. The composition of aspect 36, wherein at least one of the one or more heterologous polypeptides comprises a polypeptide that provides for enhanced localization to an SR protein granule.

Aspect 40. The composition of aspect 39, wherein the polypeptide that that provides for enhanced localization to an SR protein granule comprise an RS domain.

Aspect 41. The composition of any one of aspects 32-40, wherein the RNA-targeting CRISPR-Cas effector polypeptide exhibits reduced enzymatic activity compared to the enzymatic activity of a wild-type RNA-targeting Cas effector polypeptide.

Aspect 42. The composition of any one of aspects 32-41, wherein the RNA-targeting CRISPR-Cas effector polypeptide comprises a first higher eukaryotes and prokaryotes nucleotide-binding (HEPN) domain and a second HEPN domain.

Aspect 43. The composition of aspect 42, wherein the RNA-targeting CRISPR-Cas effector polypeptide comprises a mutation in the first HEPN domain and/or the second HEPN domain.

Aspect 44. The composition of any one of aspects 32-41, wherein the RNA-targeting CRISPR-Cas effector polypeptide is a variant Cas7-11 comprising a mutation in a catalytic domain.

Aspect 45. A method for modifying a target RNA, the method comprising contacting the target RNA with the hybrid RNA molecule of any one of aspects 1-25, wherein said contacting results in modification of the target RNA.

Aspect 46. The method of aspect 45, wherein the modification comprises inserting the RNA donor present in the hybrid RNA molecule into the target RNA.

Aspect 47. The method of aspect 45, wherein the modification comprises replacing one or more endogenous nucleotides in the target RNA with the RNA donor present in the hybrid RNA molecule.

Aspect 48. The method of any one of aspects 45-47, wherein the target RNA is in a eukaryotic cell.

Aspect 49. The method of aspect 48, wherein the eukaryotic cell is in vitro.

Aspect 50. The method of aspect 48, wherein the eukaryotic cell is in vivo.

Aspect 51. The method of any one of aspects 48-50, wherein the cell is a mammalian cell.

Aspect 52. The method of any one of aspects 45-51, wherein the target RNA is a pre-mRNA, a circular RNA, a partially spliced RNA, a non-coding RNA, a non-host cell RNA, a regulatory RNA, a coding RNA, a transfer RNA (tRNA), a pre-ribosomal RNA, a ribosomal RNA, a mature RNA with cryptic splice sites, or a long non-coding RNA (lncRNA).

Aspect 53. A method for modifying a target RNA, the method comprising contacting the target RNA with the composition of any one of aspects 30-44.

Aspect 54. The method of aspect 53, wherein the composition comprises an RNA-targeting CRISPR-Cas guide RNA comprising a targeting region that comprises a nucleotide sequence that is complementary to a second target sequence of the target RNA, wherein the RNA-targeting CRISPR-Cas guide RNA inhibits cis-splicing of the target RNA.

Aspect 55. The method of aspect 53 or aspect 54, wherein the target RNA is a pre-mRNA, a circular RNA, a partially spliced RNA, a non-coding RNA, a non-host cell RNA, a regulatory RNA, a coding RNA, a transfer RNA (tRNA), a pre-ribosomal RNA, a ribosomal RNA, a mature RNA with cryptic splice sites, or a long non-coding RNA (lncRNA).

Aspect 56. A composition comprising:
a1) a nucleic acid comprising a nucleotide sequence encoding an RNA-targeting CRISPR-Cas effector polypeptide; and
b1) a hybrid RNA molecule of any one of aspects 1-25; or
a2) a nucleic acid comprising a nucleotide sequence encoding a CRISPR-Cas fusion polypeptide comprising:
i) an RNA-targeting CRISPR-Cas effector polypeptide; and
ii) one or more heterologous polypeptides; and
b2) a hybrid RNA molecule of any one of aspects 1-25.

Aspect 57. The composition of aspect 56, wherein the nucleic acid is present in a recombinant expression vector.

Aspect 58. The composition of aspect 56 or aspect 57, wherein the nucleotide sequence encoding the RNA-targeting CRISPR-Cas effector polypeptide or the nucleotide sequence encoding the CRISPR-Cas fusion polypeptide is operably linked to a transcriptional control element.

Aspect 59. The composition of aspect 58, wherein the transcriptional control element is a regulatable promoter.

Aspect 60. A composition comprising:
a) a DNA molecule comprising:
i) a nucleotide sequence encoding an RNA-targeting CRISPR-Cas effector polypeptide; and
ii) a nucleotide sequence encoding a hybrid RNA molecule of any one of aspects 1-25; or
b) a DNA molecule comprising:
i) a nucleotide sequence encoding a fusion polypeptide comprising an RNA-targeting CRISPR-Cas effector polypeptide; and one or more heterologous polypeptides; and
ii) a nucleotide sequence encoding a hybrid RNA molecule of any one of aspects 1-25.

Aspect 61. The composition of aspect 55, wherein one or more of the nucleotide sequences is operably linked to a transcriptional control element.

Aspect 62. A method for modifying a target RNA in a cell, the method comprising introducing into the cell a composition of any one of aspects 56-61.

Aspect 63. The method of aspect 62, wherein the target RNA is in a eukaryotic cell.

Aspect 64. The method of aspect 63, wherein the eukaryotic cell is in vitro.

Aspect 65. The method of aspect 58, wherein the eukaryotic cell is in vivo.

Aspect 66. The method of any one of aspects 62-65, wherein the cell is a mammalian cell.

Aspect 67. A method of treating a trinucleotide or hexanucleotide expansion disease in an individual, the method comprising introducing into a cell in the individual a hybrid RNA molecule of any one of aspects 1-25, or a DNA molecule comprising a nucleotide sequence encoding the hybrid RNA molecule of any one of aspects 1-25, wherein the hybrid RNA molecule provides for generation of an mRNA that does not include a pathological number of trinucleotide or hexanucleotide repeats.

Aspect 68. The method of aspect 67, wherein the trinucleotide or hexanucleotide expansion disease is Huntington Disease, spinal and bulbar muscular atrophy (SBMA), spinocerebellar ataxia (SCA) Type 1, SCA Type 2, SCA Type 3, SCA Type 6, SCA Type 7, SCA Type 8, SCA Type 12, SCA Type 17, Friedreich's ataxia, C90RF72 frontotemporal dementia, Fragile X syndrome, or myotonic dystrophy.

Aspect 69. The method of aspect 67 or aspect 68, wherein the cell is a muscle cell or a neuron.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

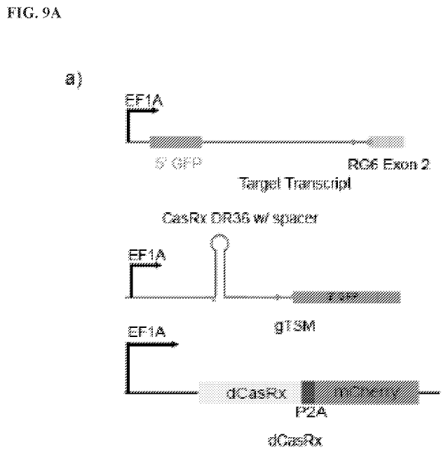

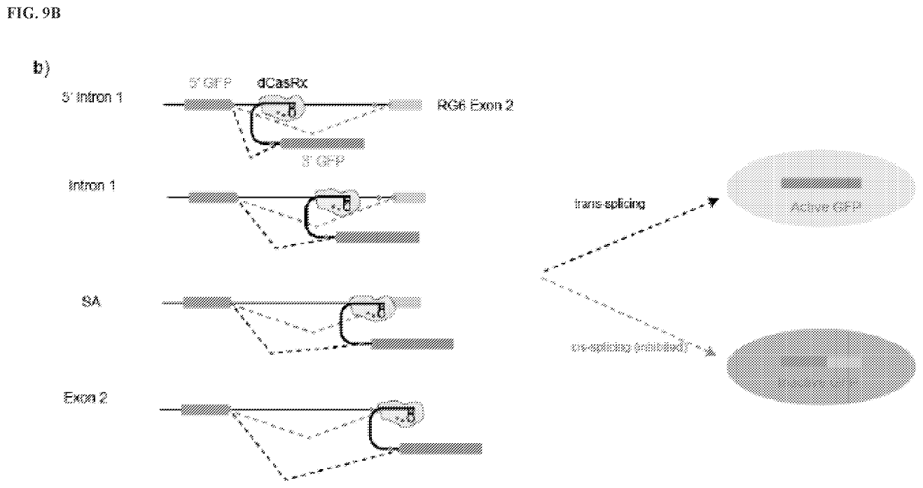

This example demonstrates 3′ trans-splicing with a trans splicing molecule (TSM) fused to a guide RNA, using a split green fluorescent protein (GFP) reporter. A TSM fused to a guide RNA is referred to as a “gTSM.” As illustrated schematically in FIG. 9A, the target transcript has 2 exons, one with the first 120 amino acids of GFP (5′ GFP) and the second that is unrelated to GFP. The gTSM targets the intron between the exons on the target transcript and trans-splices in the rest of GFP (3′ GFP) such that the trans-spliced transcript encodes active, fluorescent GFP.

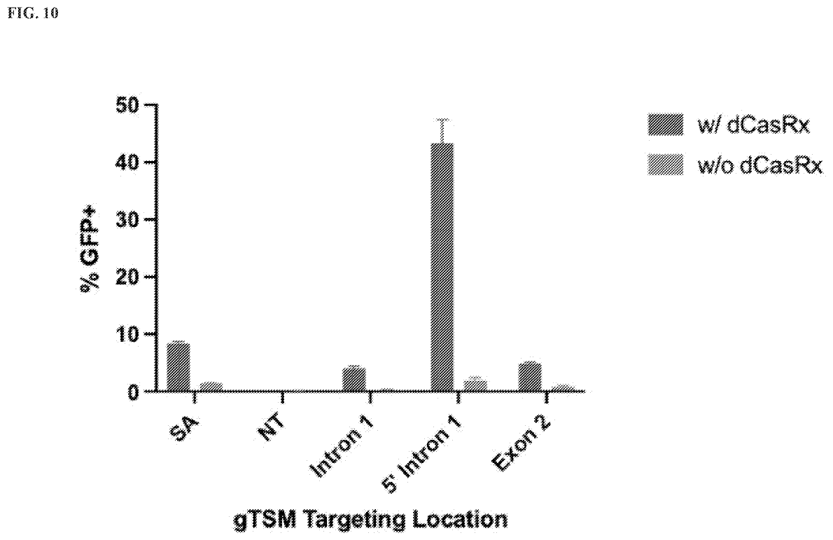

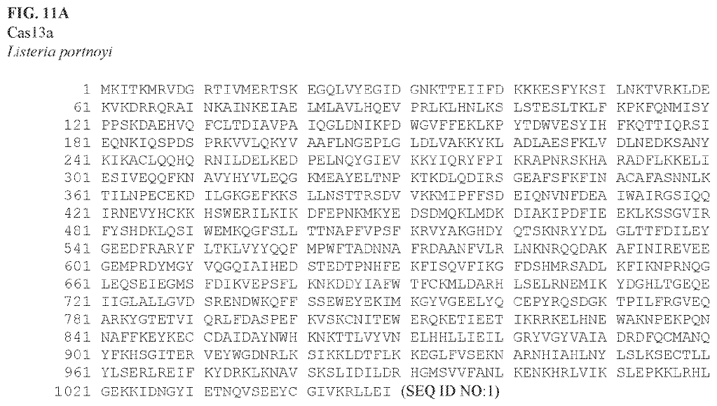

A target molecule was constructed with the 5′ half of msfGFP, RG6 intron 1, and exon 2 from the bichromatic reporter described in Orengo et al. (2006) Nucl. Acids Res. 34:e148. The gTSM constructs comprised an RfxCas13d direct repeat sequence, followed by a spacer targeting one of the locations shown in FIG. 10a or a non-targeting spacer, followed by a stuffer sequence, a branch point, a polypyrimidine tract (PPT), a splice acceptor (SA) and a cargo sequence, which in this case is the 3′ half of msfGFP. The effector plasmid consists of catalytically inactive CasRx (dCasRx) linked to mCherry with a P2A linker. All constructs are driven by the Efla promoter.

(SEQ ID NO: 64)
gttaatcgaattgaactgaaaggtattgattttaaggaggacggt
aacatactggggcacaagttggagtacaactttaacagccataat
gtgtatattaccgctgataagcagaaaaatgggataaaggccaac
tttaagatccgacataatgtcgaagatggtagtgttcaactggct
gatcattaccaacaaaatacgcccatcggagatggacctgtactc
ttgcctgacaatcattatctctccacgcaatcaaagctttccaag
gacccaaacgaaaagagagatcacatggtccttctggaatttgtg
actgccgcaggcatcactctcggtatggatgagctgtacaagtg
a.

HEK293 cells were transfected with the target transcript, gTSM, and dCasRx or pUC19.

As illustrated schematically in FIG. 9B, the gTSM included 5 different spacers (targeting sequences): 5′ Intron 1, Intron 1, Splice Acceptor (SA), Exon 2, or non-targeting (NT). The percent of GFP⁺ cells was determined.

The target and gTSM plasmids were transfected into HEK293 ft cells with and without dCasRx (FIG. 10B) at a 1:1:1 mass ratio. The results are shown in FIG. 10.

The data show strong trans-splicing activity with the gTSM targeting the 5′ end of Intron 1 of the target molecule in the presence of dCasRx, and much lower trans-splicing efficiency without the effector. The other gTSM spacers also have some trans-splicing efficiency, which is boosted in the presence of dCasRx, and there seems to be very low trans-splicing efficiency with the NT gTSM. Overall, a spacer sequence dependent and effector dependent effect was observed on trans-splicing efficiency.

As shown in FIG. 10, the pUC19 control (without (w/o) dCasRx) did not provide for production of a GFP-encoding transcript. In the cells that included dCasRx, a low level of background trans-splicing was observed, as illustrated with the cells transfected with the NT gTSM. The best gTSM targeting location was at the beginning of the intron on the target transcript (5′ Intron 1), which resulted in ˜40% GFP⁺ cells.

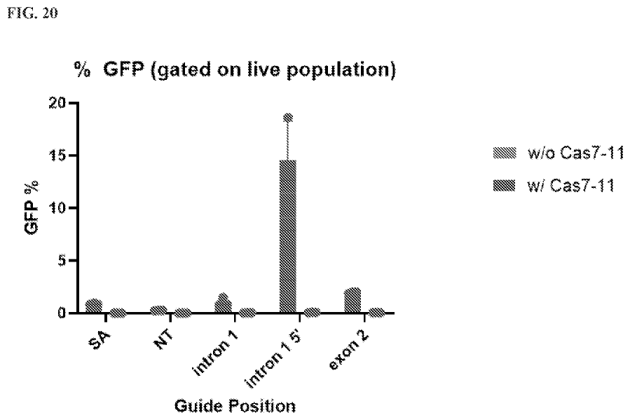

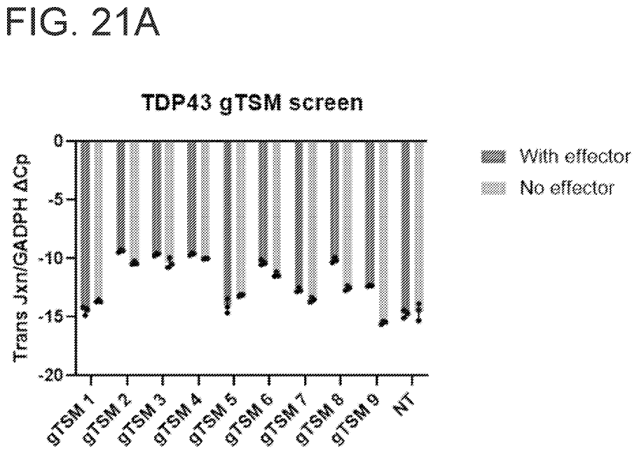

This result was also observed when the effector was catalytically active Cas7-11 (FIG. 20). In this case, the absence of the effector abrogates trans-splicing efficiency. This points to the sensitivity of the gTSM itself to flanking regions, as the direct repeat sequencing in the gTSMs used with Cas7-11 is different from the CasRx direct repeat.

### Example 2

A “gTSM screen” and a “gIM screen” were carried out. For the gTSM screen, multiple gTSMs targeting various locations within an intron of a transcript were screened; this was done using a 2-plasmid transfection (gTSM construct and effector construct). For the gIM screen, multiple gIMs targeting various locations within the intron of a transcript downstream of the gTSM, identified in the gTSM screen as having the highest activity, were screened; this was done using a 3-plasmid transfection (gTSM construct, gIM construct, effector construct).

The qPCR data indicated the levels of trans-splicing junctions relative to GAPDH expression. The closer to the x-axis indicated a higher level of trans-splicing. Droplet digital PCR (ddPCR) directly quantified cis and trans junctions; efficiencies are reported as a percentage of total cis and trans junctions.

### Methods

### Cell Culture

The HEK293 ft cell line was purchased from ThermoFisher. HEK293 ft was cultured in DMEM, supplemented with fetal bovine serum (FBS) with 100 U/mL of Penicillin-Streptomycin.

### Plasmid Transfections

All plasmids were prepared using the NucleoSpin Xtra Midi or Maxi EF kits. Hek293t cells were seeded at 14k density into 96 well plates (ThermoFisher 152037). Plasmids were transfected using Lipofectamine 2000 from Invitrogen according to the manufacturer's protocol.

### Flow Cytometry

All flow cytometry was performed using an Attune NxT Flow Cytometer. Cells were trypsinized using TrypLE Express reagent according to the manufacturer's protocols 48 hours post transfection. Trypsinization was inhibited by adding Stain Buffer with 2% FBS (Catalog #: 554656). Cells were transferred to a clear, 96 well U bottom plate, spun for 5 minutes @500 rpm. Trypsin and stain buffer were removed, and 200 μL of fresh stain buffer was added to each well and mixed. The cells were then run on the Attune according to manufacturer protocols.

qPCR Readout of Endogenous Trans-Splicing

48 hours post-transfection, transfected cells were washed with DPBS (ThermoFisher, 14190094). Cells were then incubated and mixed for 8 minutes at room temperature with 50 μL of RNA lysis buffer (9.6 mM Tris-HCl (pH 7.8), 0.5 mM MgCl2, 0.44 mM CaCl2, 10 M DTT, 0.1% (wt/vol) Triton X-114, 300 U ml-1 DNAse I, and 3 U ml-1 proteinase K in UltraPure water). 30 μL of lysed cells were then transferred into a PCR plate with 3 μL of RNA lysis stop solution (1 mM Proteinase K inhibitor, 90 mM EGTA, 113 μM DTT in UltraPure water) and incubated at room temperature for 2 minutes. 8 μL of the lysis solution was then transferred into the RevertAid RT Reverse Transcription mastermix (Thermo Fisher Scientific, cat. No. K1691) and the reverse transcription reaction was incubated at 25° C. for 10 min, 37° C. for 60 min and then 95° C. for 5 min. The completed reverse transcription reaction was then carried into the qPCR reaction. qPCR primers were designed using PrimerBlast and probes were designed to bind across the junction of the trans or cis splice junctions. The RT reaction was then added as a template into a mastermix for four technical replicates using Applied Biosystems™ TaqMan™ Universal PCR Master Mix (Supplier: Applied Biosystems™ 4304437) and read out on a Roche LightCycler. For analysis, trans-splicing junction Cp values were normalized to the corresponding GAPDH Cp values for each condition to account for cell count.

ddPCR Readout of Endogenous Trans-Splicing

Transfected cells were lysed and reverse transcribed as described previously. qPCR primers were designed using PrimerBlast and probes were designed to bind across the junction of the trans or cis splice junctions. qPCR reactions were set up according to the manufacturer's instructions using BioRad ddPCR SuperMix for Probes (#1863024). Droplets were generated with the BioRad Automated Digital Droplet Generator per manufacturer instructions and read out using the BioRad Droplet Reader. Trans-splicing efficiency was measured as a percentage of the sum of trans and cis splicing junctions.

### Results

A guided trans-splicing molecule (gTSM) and a guided interfering molecule (gIM) were used for CRISPR-catalyzed trans-splicing, targeting an exon of various target genes.

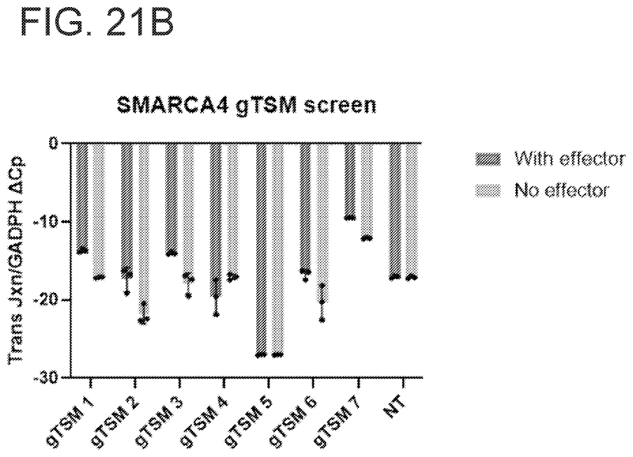

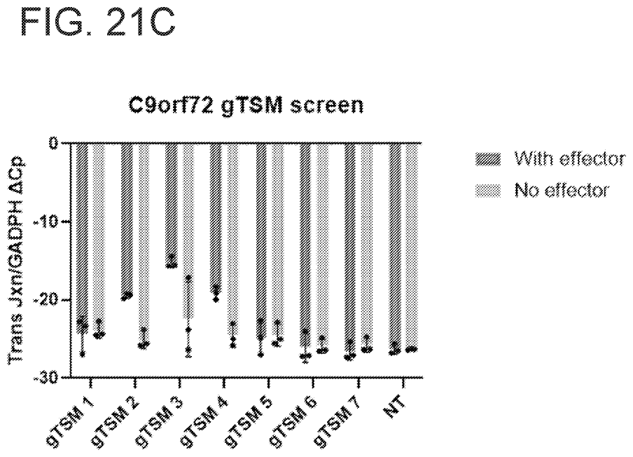

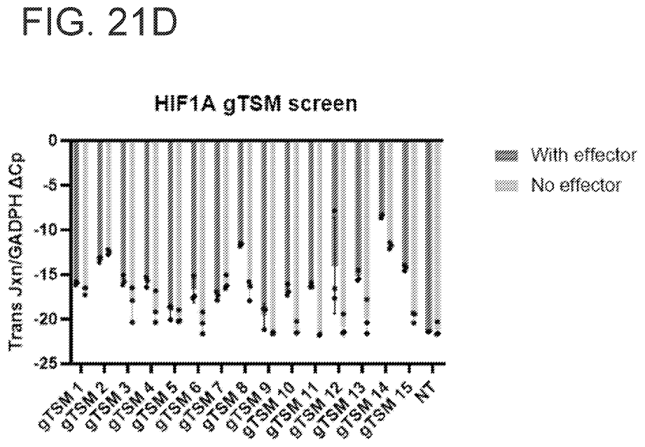

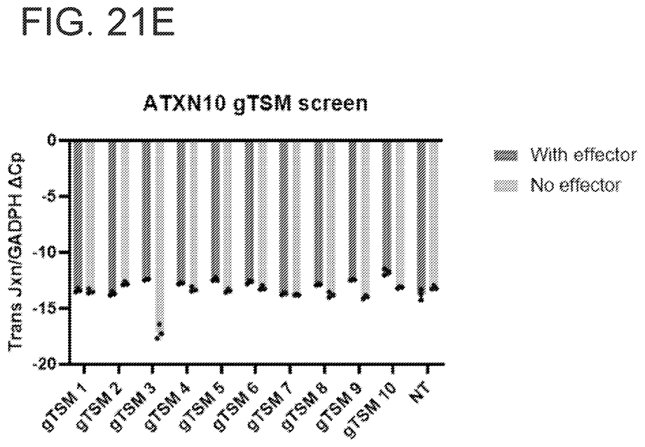

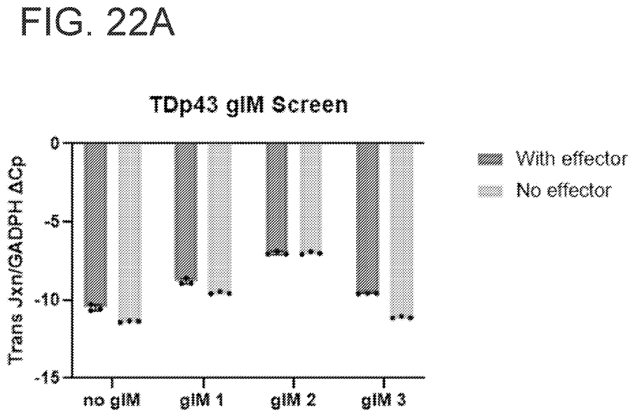

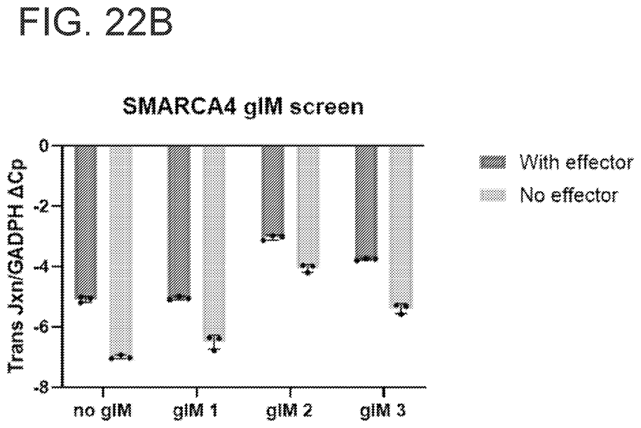

The results are shown in FIG. 21 and FIG. 22.

FIG. 21A-21E. ΔCp of trans-splicing junctions over a set of endogenous genes for testing gTSMs. The gTSM constructs were tested across a set of genes using dCasRx as the effector: TDP43 (TAR DNA-binding protein-43), SMARCA4 (SWI/SNF related, matrix associated, actin dependent regulator of chromatin, subfamily A, member 4), C9orf72 (chromosome 9 open reading frame 72), HIF1A (hypoxia inducible factor 1 subunit alpha), and ATXN10 (spinocerebellar ataxia type 10 protein). Each transfection consisted of two constructs, one construct expressed the gTSM and the other expressed the effector (dCasRx) FIG. 21A (targeting TDP43). Various sites on a TDP43 intron were targeted with a gTSM. FIG. 21B (targeting SMARCA4). Various sites on a SMARCA4 intron were targeted with a gTSM. FIG. 21C (targeting C9orf72). Various sites on a C9orf72 intron were targeted with a gTSM. FIG. 21D (targeting HIF1A). Various sites on a HIF1A intron were targeted with a gTSM.

FIG. 21E (targeting ATXN10). Various sites on an ATXN10 intron were targeted with a gTSM.

The best performing gTSM (from FIG. 21A-21E) was chosen for two of these genes (TDP43, SMARCA4) and were subsequently used to test the gIM concept. The results are shown in FIG. 22A-22B. gTSM-2 from the gTSM screen targeting TDP43 was paired with different gIMs targeting downstream of the gTSM on the same intron. gTSM-7 from the gTSM screen targeting SMARCA4 was paired with different gIMs targeting downstream of the gTSM on the same intron These transfections consisted of three constructs: one construct expressed the gTSM, a second construct expressed the effector (dCasRx), and the third construct expressed Cas7-11 and its guide.

FIG. 22A-22B. Targeting TDP43 with hit gTSM and testing gIMs (FIG. 22A). Targeting SMARCA4 with hit gTSM and testing gIMs (FIG. 22B).

### Example 3

This example demonstrates 3′ trans-splicing of an endogenous transcript using a trans-splicing molecule (TSM) fused to a guide RNA, referred to as a “gTSM”, and a guided interfering molecule, referred to as a “gIM”. The gTSM comprises a trans-splicing molecule comprising a spacer targeting the intron intervening exons 7 and 8 of the ITGB1 (integrin subunit beta 1) transcript or a non-targeting spacer, a cargo sequence (i.e. donor RNA), and, optionally, an RNA-targeting CRISPR effector. The gIM comprises an RNA-targeting CRISPR effector and an interfering guide that targets downstream of the gTSM for cleavage.

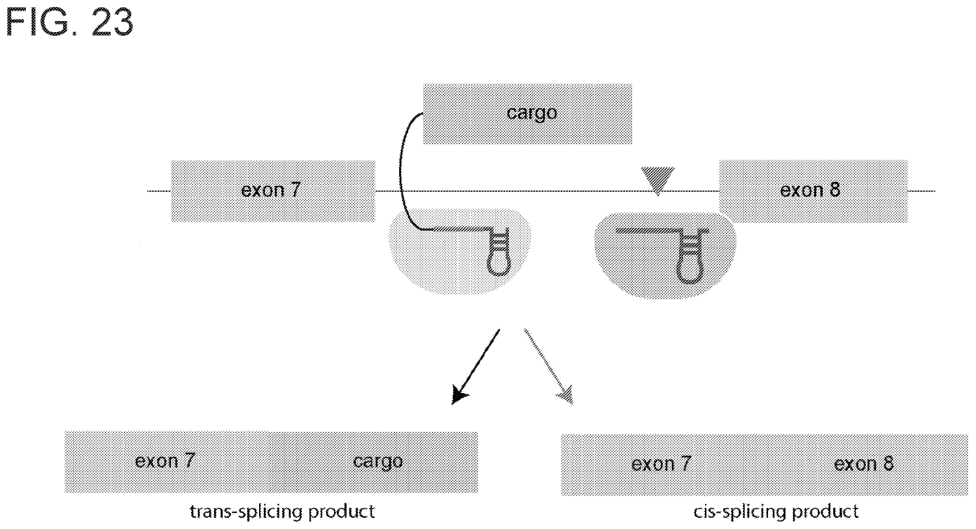

As illustrated schematically in FIG. 23, the gTSM and gIM target the intron between exon 7 and 8 of the target ITGB1 transcript and trans-splices in the gTSM cargo sequence, resulting in a trans-spliced ITGB1 transcript with exon 7 fused to the cargo and no exon 8.

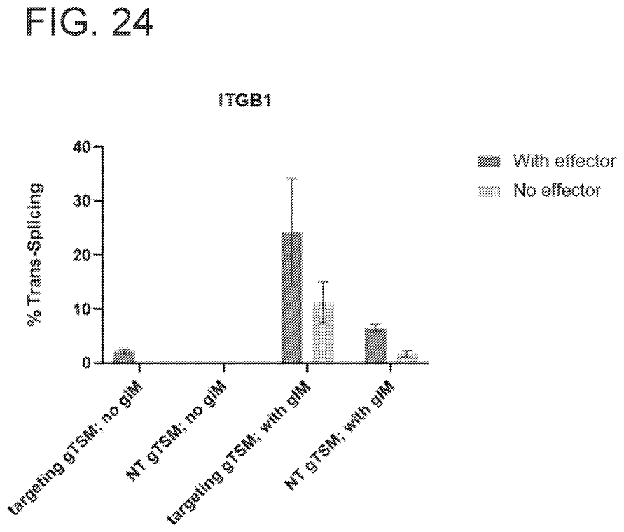

Four approaches for trans-splicing of the ITGB1 transcript were tested: trans-splicing with only a gTSM comprising a targeting spacer (targeting gTSM; no gIM), trans-splicing with both a gTSM comprising a targeting spacer and the gIM (targeting gTSM; with gIM), trans-splicing with both a gTSM comprising a non-targeting spacer and the gIM (NT gTSM; with gIM), and a gTSM comprising a non-targeting spacer only (NT gTSM; no gIM) as a control. Each of these approaches was also tested with or without an RNA-targeting CRISPR effector for the gTSM. Trans-splicing in ITGB1 (integrin subunit beta 1) was measured with ddPCR to quantitatively assess trans-splicing efficiencies. The results are shown in FIG. 24.

As shown in FIG. 24, the condition with both a targeting gTSM and gIM (targeting gTSM; with gIM) resulted in ˜20% trans-splicing. The results demonstrate ˜2% trans-splicing efficiency without gIM (targeting gTSM; no gIM).

FIG. 24. Percent trans-splicing efficiency for ITGB1 measured by ddPCR.

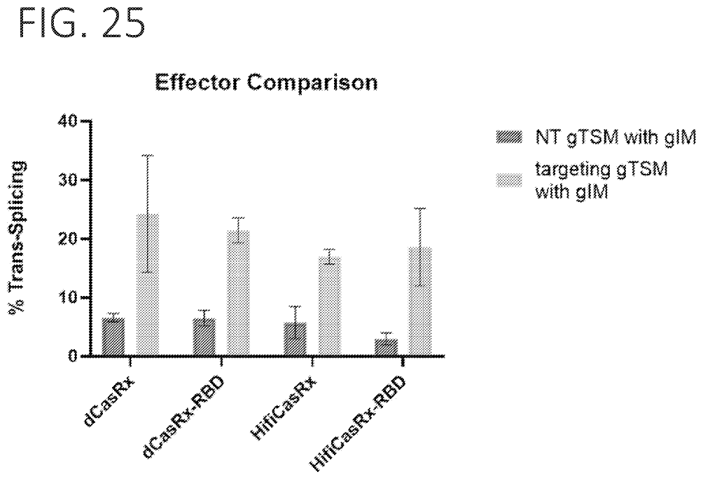

The Trans-splicing efficiencies for the ITGB1 transcript of four different RNA-targeting CRISPR effectors were tested: trans-splicing with dCasRx, trans-splicing with dCasRx fused to a double-stranded RNA-binding domain (dCASRx-RBD), trans-splicing with an enhanced dCasRx (HifiCasRx), and trans-splicing with an enhanced dCasRx fused to a double-stranded RNA-binding domain (HifiCasRx-RBD). Each RNA-targeting CRISPR effector was tested with the combination of a targeting gTSM with a gIM or the combination of a non-targeting gTSM (NT gTSM) with a gIM. Trans-splicing in ITGB1 was measured with ddPCR to quantitatively assess trans-splicing efficiencies. The results are shown in FIG. 25.

As shown in FIG. 25, each RNA-targeting CRISPR effector, in combination with a targeting gTSM and gIM, demonstrated ˜15%-20% trans-splicing of ITGB1 transcripts.

FIG. 25. Percent trans-splicing efficiency for ITGB1 with various RNA-targeting CRISPR effectors measured by ddPCR.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A hybrid RNA molecule comprising
a) a targeting region that comprises a nucleotide sequence that is complementary to a target sequence of a target RNA; and
b) a donor sequence to said targeting region.

2. The hybrid RNA molecule of claim 1, further comprising one or more of:
a) a binding region that binds to an RNA binding or modifying polypeptide;
b) a protein-recruiting region;
c) a stabilization region.

3. The hybrid RNA molecule of claim 2, wherein the hybrid RNA molecule comprises a binding region that binds to an RNA binding or modifying polypeptide, and wherein the RNA binding or modifying polypeptide is an RNA-targeting CRISPR-Cas effector polypeptide.

4. The hybrid RNA molecule of claim 3, wherein the RNA-targeting CRISPR-Cas effector polypeptide is a Type II, a Type III, or a Type VI CRISPR-Cas effector polypeptide.

5. The hybrid RNA molecule of claim 4, wherein the Type II CRISPR-Cas effector polypeptide is a Cas9 polypeptide.

6. The hybrid RNA molecule of claim 4, wherein the Type VI CRISPR-Cas effector polypeptide is a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide.

7. The hybrid molecule of claim 4, wherein the Type III RNA-targeting CRISPR-Cas effector polypeptide is a Cas7-11 polypeptide.

8. The hybrid RNA molecule of claim 2, wherein the hybrid RNA molecule comprises a protein-recruiting region.

9. The hybrid RNA molecule of claim 8, wherein the protein-recruiting region comprises a nucleotide sequence that binds one or more spliceosome polypeptides.

10. The hybrid RNA molecule of claim 2, wherein the hybrid RNA molecule comprises a stabilization region.

11. The hybrid RNA molecule of claim 10, wherein the stabilization region is capable of forming a secondary structure.

12. The hybrid RNA molecule of claim 11, wherein the secondary structure comprises a pseudoknot, a stem-loop, or a tetraloop.

13. The hybrid RNA molecule of any one of claims 1-12, wherein the RNA donor comprises one or more of:
a) a coding region comprising a nucleotide sequence encoding all or a portion of a polypeptide;
b) an untranslated region (UTR);
c) a bar code;
d) an internal ribosomal entry site (IRES);
e) a poly(pyrimidine) tract;
f) an RNA response element;
g) an intron; and
h) an aptamer.

14. The hybrid RNA molecule of any one of claims 1-13, further comprising a splice function region comprising one or more of:
a) a splice modifying sequence;
b) a splice exclusion sequence;
c) an intron;
d) a branch point;
e) an intronic splice enhancer;
f) an intronic splice silencer; and
g) a poly(pyrimidine tract).

15. The hybrid DNA molecule of claim 14, wherein the splice modifying sequence comprises a splice acceptor, a splice donor, a branchpoint, or a polypyrimidine tract.

16. The hybrid RNA molecule of claim 13, wherein the RNA donor comprises a nucleotide sequence encoding all or a portion of a polypeptide.

17. The hybrid RNA molecule of claim 16, wherein the RNA donor comprises a nucleotide sequence encoding a portion of a non-pathological form of a disease-associated polypeptide, wherein the disease-associated polypeptide comprises an amino acid expansion.

18. The hybrid RNA molecule of claim 17, wherein the disease-associated polypeptide comprises a poly(Gln) repeat region, and wherein the non-pathological form of the polypeptide comprises a (Gln)x repeat region, wherein x is less than the number of repeats associated with a disease.

19. The hybrid RNA molecule of claim 17, wherein the disease-associated polypeptide comprises a poly(Glu) repeat region, and wherein the non-pathological form of the polypeptide comprises a (Glu)x repeat region, wherein x is less than the number of repeats associated with a disease.

20. The hybrid RNA molecule of claim 13, wherein the RNA donor comprises an intron comprising a non-pathological number of repeats of GAA.

21. The hybrid RNA molecule of claim 13, wherein the RNA donor comprises a 5′-UTR comprising a non-pathological number of repeats of CGG, CCG, or GGC.

22. The hybrid RNA molecule of claim 13, wherein the RNA donor comprises a 3′-UTR comprising a non-pathological number of repeats of CTG.

23. The hybrid RNA molecule of any one of claims 1-22, comprising a SINE-derived nuclear RNA Localization (SIRLOIN) motif.

24. The hybrid RNA molecule of any one of claims 1-23, further comprising a second targeting region that comprises a nucleotide sequence that is complementary to a second target sequence of the target RNA and that interferes with cis splicing of the target RNA.

25. The hybrid RNA molecule of any one of claims 1-24, wherein the RNA donor has a length of from 2 nucleotides to 10,000 nucleotides.

26. A DNA molecule comprising a nucleotide sequence encoding the hybrid RNA molecule of any one of claims 1-25.

27. The DNA molecule of claim 26, wherein the nucleotide sequence is operably linked to a transcriptional control element.

28. The DNA molecule of claim 27, wherein the transcriptional control element comprises a promoter, and wherein the promoter is one or more of: a constitutive promoter, an inducible promoter, a cell type-specific promoter, and a tissue-specific promoter.

29. A recombinant expression vector comprising the DNA molecule of any one of claims 26-28.

30. A composition comprising:
a) the hybrid RNA molecule of any one of claims 1-25; and
b) one or more of a nanoparticle, a lipid, a buffer, and a nuclease inhibitor.

31. A composition comprising:
al) an RNA-targeting CRISPR-Cas effector polypeptide; and
b1) a hybrid RNA molecule of any one of claims 1-25; or
a2) a fusion polypeptide comprising:
i) an RNA-targeting CRISPR-Cas effector polypeptide; and
ii) one or more heterologous polypeptides; and
b2) a hybrid RNA molecule of any one of claims 1-25.

32. The composition of claim 31, wherein the RNA-targeting CRISPR-Cas effector polypeptide is a Type II, a Type III, or a Type VI CRISPR-Cas effector polypeptide.

33. The composition of claim 32, wherein the Type II CRISPR-Cas effector polypeptide is a Cas9 polypeptide.

34. The composition of claim 32, wherein the Type III CRISPR-Cas effector polypeptide is a Cas7-11 polypeptide.

35. The composition of claim 32, wherein the Type VI CRISPR-Cas effector polypeptide is a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide.

36. The composition of claim 31, wherein the composition comprises:
a) a fusion polypeptide comprising:
i) an RNA-targeting CRISPR-Cas effector polypeptide; and
ii) one or more heterologous polypeptides; and
b) a hybrid RNA molecule of any one of claims 1-25.

37. The composition of claim 36, wherein at least one of the one or more heterologous polypeptides comprises an RNA Polymerase II-binding polypeptide.

38. The composition of claim 37, wherein the RNA Polymerase II-binding polypeptide comprises a C-terminal interacting domain (CID).

39. The composition of claim 36, wherein at least one of the one or more heterologous polypeptides comprises a polypeptide that provides for enhanced localization to an SR protein granule.

40. The composition of claim 39, wherein the polypeptide that that provides for enhanced localization to an SR protein granule comprise an RS domain.

41. The composition of any one of claims 32-40, wherein the RNA-targeting CRISPR-Cas effector polypeptide exhibits reduced enzymatic activity compared to the enzymatic activity of a wild-type RNA-targeting Cas effector polypeptide.

42. The composition of any one of claims 32-41, wherein the RNA-targeting CRISPR-Cas effector polypeptide comprises a first higher eukaryotes and prokaryotes nucleotide-binding (HEPN) domain and a second HEPN domain.

43. The composition of claim 42, wherein the RNA-targeting CRISPR-Cas effector polypeptide comprises a mutation in the first HEPN domain and/or the second HEPN domain.

44. The composition of any one of claims 32-41, wherein the RNA-targeting CRISPR-Cas effector polypeptide is a variant Cas7-11 comprising a mutation in a catalytic domain.

45. A method for modifying a target RNA, the method comprising contacting the target RNA with the hybrid RNA molecule of any one of claims 1-25, wherein said contacting results in modification of the target RNA.

46. The method of claim 45, wherein the modification comprises inserting the RNA donor present in the hybrid RNA molecule into the target RNA.

47. The method of claim 45, wherein the modification comprises replacing one or more endogenous nucleotides in the target RNA with the RNA donor present in the hybrid RNA molecule.

48. The method of any one of claims 45-47, wherein the target RNA is in a eukaryotic cell.

49. The method of claim 48, wherein the eukaryotic cell is in vitro.

50. The method of claim 48, wherein the eukaryotic cell is in vivo.

51. The method of any one of claims 48-50, wherein the cell is a mammalian cell.

52. The method of any one of claims 45-51, wherein the target RNA is a pre-mRNA, a circular RNA, a partially spliced RNA, a non-coding RNA, a non-host cell RNA, a regulatory RNA, a coding RNA, a transfer RNA (tRNA), a pre-ribosomal RNA, a ribosomal RNA, a mature RNA with cryptic splice sites, or a long non-coding RNA (lncRNA).

53. A method for modifying a target RNA, the method comprising contacting the target RNA with the composition of any one of claims 30-44.

54. The method of claim 53, wherein the composition comprises an RNA-targeting CRISPR-Cas guide RNA comprising a targeting region that comprises a nucleotide sequence that is complementary to a second target sequence of the target RNA, wherein the RNA-targeting CRISPR-Cas guide RNA inhibits cis-splicing of the target RNA.

55. The method of claim 53 or claim 54, wherein the target RNA is a pre-mRNA, a circular RNA, a partially spliced RNA, a non-coding RNA, a non-host cell RNA, a regulatory RNA, a coding RNA, a transfer RNA (tRNA), a pre-ribosomal RNA, a ribosomal RNA, a mature RNA with cryptic splice sites, or a long non-coding RNA (lncRNA).

56. A composition comprising:
al) a nucleic acid comprising a nucleotide sequence encoding an RNA-targeting CRISPR-Cas effector polypeptide; and
b1) a hybrid RNA molecule of any one of claims 1-25; or
a2) a nucleic acid comprising a nucleotide sequence encoding a CRISPR-Cas fusion polypeptide comprising:
i) an RNA-targeting CRISPR-Cas effector polypeptide; and
ii) one or more heterologous polypeptides; and
b2) a hybrid RNA molecule of any one of claims 1-25.

57. The composition of claim 56, wherein the nucleic acid is present in a recombinant expression vector.

58. The composition of claim 56 or claim 57, wherein the nucleotide sequence encoding the RNA-targeting CRISPR-Cas effector polypeptide or the nucleotide sequence encoding the CRISPR-Cas fusion polypeptide is operably linked to a transcriptional control element.

59. The composition of claim 58, wherein the transcriptional control element is a regulatable promoter.

60. A composition comprising:
a) a DNA molecule comprising:
i) a nucleotide sequence encoding an RNA-targeting CRISPR-Cas effector polypeptide; and
ii) a nucleotide sequence encoding a hybrid RNA molecule of any one of claims 1-25; or
b) a DNA molecule comprising:
i) a nucleotide sequence encoding a fusion polypeptide comprising an RNA-targeting CRISPR-Cas effector polypeptide; and one or more heterologous polypeptides; and
ii) a nucleotide sequence encoding a hybrid RNA molecule of any one of claims 1-25.

61. The composition of claim 55, wherein one or more of the nucleotide sequences is operably linked to a transcriptional control element.

62. A method for modifying a target RNA in a cell, the method comprising introducing into the cell a composition of any one of claims 56-61.

63. The method of claim 62, wherein the target RNA is in a eukaryotic cell.

64. The method of claim 63, wherein the eukaryotic cell is in vitro.

65. The method of claim 58, wherein the eukaryotic cell is in vivo.

66. The method of any one of claims 62-65, wherein the cell is a mammalian cell.

67. A method of treating a trinucleotide or hexanucleotide expansion disease in an individual, the method comprising introducing into a neuron in the individual a hybrid RNA molecule of any one of claims 1-25, or a DNA molecule comprising a nucleotide sequence encoding the hybrid RNA molecule of any one of claims 1-25, wherein the hybrid RNA molecule provides for generation of an mRNA that does not include a pathological number of trinucleotide or hexanucleotide repeats.

68. The method of claim 67, wherein the trinucleotide or hexanucleotide expansion disease is Huntington Disease, spinal and bulbar muscular atrophy (SBMA), spinocerebellar ataxia (SCA) Type 1, SCA Type 2, SCA Type 3, SCA Type 6, SCA Type 7, SCA Type 8, SCA Type 12, SCA Type 17, Friedreich's ataxia, C90RF72 frontotemporal dementia, Fragile X syndrome, or myotonic dystrophy.

69. The hybrid RNA molecule of claim 16, wherein the RNA donor comprises a nucleotide sequence encoding a fluorescent protein.

70. The hybrid RNA molecule of claim 16, wherein the RNA donor comprises a nucleotide sequence encoding an epitope tag.
